(19) United States
(12) Patent Application Publication
(10) Pub. No.: US 2021/0015708 A1
(43) Pub. Date: Jan. 21, 2021

(54) TWO-COMPONENT-MIXING PREFILLED SYRINGE KIT

(71) Applicant: CMC-PHARMA CO., LTD, Osaka-shi, Osaka (JP)

(72) Inventors: Shuji MORIMOTO, Suita-shi, Osaka (JP); Hirosi YAMAUTI, Kawanishi-shi, Hyogo (JP); Jyotaro KISHIMOTO, Nishinomiya-shi, Hyogo (JP)

(21) Appl. No.: 17/041,452

(22) PCT Filed: Mar. 27, 2019
(86) PCT No.: PCT/JP2019/013354

§ 371 Date: Sep. 25, 2020

(30) Foreign Application Priority Data
Mar. 28, 2018 (JP) 2018-062038

(51) Int. Cl. A61J 1/20; A61M 5/28; A61M 5/32; A61J 1/14

(52) U.S. Cl. CPC A61J 1/2096; A61J 1/1406; A61M 5/3202; A61M 5/285

(57) ABSTRACT

In a two-component-mixing prefilled syringe kit for preparing an injection solution by mixing a drug component in a syringe barrel and a liquid component in a container, when the injection needle pierced on the seal member of the container outlet is pulled out after preparation of the injection solution, a leak to the outside of the remaining liquid in the container is effectively prevented.

Comprise a syringe barrel in which an injection needle is attached on one end, a plunger is inserted slidably through the opening on the other end, and the solid or liquid component constituting the injection solution is filled, and a container in which an outlet is sealed by a seal member that can be pierced by the injection needle, and the liquid component constituting the injection solution is filled, and a holding member which holds the syringe barrel so that the syringe barrel is movable along the axial direction with the tip of the injection needle facing the outlet of the container, and a space-forming body which forms a space facing the surface of the seal member in a space sealed from the outside, and the space-forming body is formed of a material that can be pierced by the injection needle at the portion facing the tip of the injection needle along the barrel axis direction of the syringe barrel.

### TECHNICAL FIELD

This invention relates to a two-component-mixing prefilled syringe kit in which a syringe barrel filled with one component constituting an injection solution and a container filled with the other component of the injection solution are arranged to face each other, and the component in the syringe barrel and the component in the container are mixed to prepare the injection solution.

### BACKGROUND ART

According to Patent document 1 related to a conventional prefilled syringe kit, a structure is known in which a liquid component is filled inside a syringe barrel, and an injection needle attached to the tip of the syringe barrel is arranged to face and be separated from a rubber seal member at the outlet of a container filled with a drug, and the syringe barrel and the drug container are connected by a nesting mechanism so as to be relatively movable in the barrel axis direction of the syringe barrel. In Patent document 1, during use, the syringe barrel is pushed toward the drug container side, the tip of the injection needle is pierced through the seal member, the inside of the syringe barrel and the inside of the drug container are communicated, and then a plunger inserted into the rear end opening of the syringe barrel is pushed to inject the liquid component inside the syringe barrel into the drug container to mix the drug and the liquid, and a prepared injection solution is returned to the inside of the syringe barrel.

Also, according to another Patent document 2, a structure is known in which a powder drug is filled in a syringe barrel, and a tip of an injection needle attached to a tip of the syringe barrel is arranged to face and be pierced (but, in non-penetrating manner) by a seal member at an outlet of a container filled with a liquid component, and the syringe barrel and the liquid container are connected so as to be relatively movable in the axial direction of the syringe barrel by a connection barrel. In the prefilled syringe kit of Patent document 2, during use, a plunger inserted into the rear end opening of the syringe barrel is pushed to allow the tip of the injection needle to penetrate the seal member, thereby the inside of the syringe barrel and the inside of the liquid container are communicated, pressurized air is sent to the inside of the liquid container, and the liquid component in the container is moved to the syringe barrel side by the pressurized air to mix the drug and the liquid component to prepare an injection solution.

### PRECEDING TECHNICAL DOCUMENTS

### Patent Documents

Patent document 1: JP S59-118164 A

Patent document 2: JP H05-31189 A

### SUMMARY OF THE INVENTION

### Problems to be Resolved by the Invention

In the prefilled syringe kit described in Patent document 1, the syringe barrel, the plunger, the drug container and the like are exposed to the outside. In the prefilled syringe kit described in Patent document 2, the syringe barrel and the plunger are covered by a cover attached to the rear edge of the connecting barrel, but the liquid container, the connecting portion between the connecting barrel and the liquid container, and the like are exposed to the outside. Therefore, in the conventional prefilled syringe kit, during storage and preparation of the injection solution, there is a possibility that the drug component, the liquid component, or the like filled inside leaks to the outside. In particular, in the case that the injection needle penetrates the seal member of the liquid container to transfer the liquid inside the container to the side of the syringe barrel and mix it with the component in the syringe barrel to prepare the injection solution, after the preparation of the injection solution, when the injection needle is pulled out from the container, the liquid component remaining in the container may leak to the outside through the needle piercing place of the seal member.

In view of the above problems, an object of the present invention is to provide a two-component-mixing prefilled syringe kit in which a leak of the liquid component remaining in the container to the outside can be effectively prevented when preparing the injection solution by mixing the drug component in the syringe barrel and the liquid component in the container.

### Means of Solving the Problems

A two-component-mixing prefilled syringe kit according to the present invention comprises; a syringe barrel in which an injection needle is attached to one end, a plunger is slidably inserted from an opening on the other end, and a solid component or a liquid component constituting an injection solution is filled inside; a container in which an outlet is sealed with a seal member that can be pierced by the injection needle, and a liquid component constituting the injection solution is filled inside; and a holding member for holding at least one of the syringe barrel or the container so as to be movable along the barrel axis direction of the syringe barrel with a tip of the injection needle facing the outlet of the container; wherein, it is characterized that a space-forming body which forms a space facing a surface of the seal member with a space sealed from the outside is arranged so as to be in close contact with the surface of the seal member, and the space-forming body is formed of a material that can be pierced by the injection needle at a portion facing the tip of the injection needle along the barrel axis direction of the injection barrel.

In the present invention, at least one of the syringe barrel or the container held by the holding member with the tip of the injection needle facing the outlet of the container moves along the barrel axis direction of the syringe barrel so that the container and the syringe barrel approach each other, and the tip of the injection needle attached to one end of the syringe barrel pierces the portion of the space-forming body and the seal member of the outlet of the container that face along the barrel axis direction, and after the inside of the container and the inside of the syringe barrel have communicated, the plunger slidably inserted from the other end opening of the syringe barrel is operated to push the air in the syringe barrel into the container inside, then the liquid component in the container is moved into the syringe barrel by the pressurized air in the container, mixing with the solid component or liquid component in the syringe barrel so as to prepare the injection solution. The injection needle pierced with the seal member of the container is pulled out from the seal member in order to separate the syringe barrel containing the prepared injection solution from the container. At this time, the liquid component remaining in the container may leak to the surface of the seal member from a needle piercing place of the seal member which the injection needle has got out, but the liquid component does not leak outside because the surface of the seal member is exposed to the space sealed from the outside.

A volume of the space surrounded by the surface of the seal member and the space-forming body is in the range of 0.001 ml to 0.03 ml.

Because, if it is less than 0.001 ml, there is a possibility that the amount of liquid leaking from the surface of the seal member may exceed 0.001 ml, while if it is more than 0.03 ml, the size of the space-forming body becomes unnecessarily large.

The two-component-mixing prefilled syringe kit according to the present invention is characterized that it comprises a needle cap which is formed of a material that can be pierced by the injection needle to cover the injection needle from the tip side, and fixed at the base end side to the base of the injection needle or the outer surface of the syringe barrel, and the needle cap has a tubular portion in which a tube axis direction is along to the direction toward the seal member and an expected piercing place by the injection needle is located at the inner side, on a front portion facing the seal member, and the space-forming body is constituted by the front portion of the needle cap and the tubular portion in close contact with the surface of the seal member.

In the present invention, when the container and the syringe barrel are approached each other and the tubular portion attached on the front portion of the needle cap facing the seal member of the container is pushed against the seal member, a space which the surface of the seal member faces is formed of a space sealed from the outside by the front portion and the tubular portion of the needle cap. Furthermore, the tip of the injection needle is passed through the inside of the tubular portion from the expected piercing place at the front portion of the needle cap to pierce the seal member of the container outlet, the inside of the container and the inside of the syringe barrel communicate, then moving the liquid component in the container into the syringe barrel, the injection solution is prepared. When pulling out the injection needle pierced with the seal member of the container from the seal member in order to separate the syringe barrel containing the prepared injection solution from the container, the liquid component in the container may leaks to the surface of the seal member, but since the space which the surface of the seal member faces is sealed from the outside by the front portion of the needle cap and the tubular portion pushed against the seal member, the liquid component does not leak to the outside.

The two-component-mixing prefilled syringe kit according to the present invention is characterized that the needle cap has a flange portion being in a direction intersecting with the barrel axis direction of the syringe barrel on an outer side surface of the front portion, and the holding member has a mount portion for mounting the syringe barrel, a pushing portion for pushing the flange portion against the container when moving to the container side along the barrel axis direction of the syringe barrel, and a lock part for holding the state where the pushing portion is pushing the flange portion against the container.

In the present invention, the holding member with the syringe barrel mounted on the mount portion is moved toward the container side along the barrel axis direction so that the tip of the injection needle pierces the front portion of the needle cap and the seal member of the container, and when the flange portion on the outer side surface of the front portion of the needle cap intersecting with the barrel axis direction of the syringe barrel is pushed against the container by the pushing portion provided on the holding member, the state where the pushing portion is pushing the flange portion against the container is held by the lock part provided on the holding member. As a result, the tip of the injection needle pierces the front portion of the needle cap and the seal member of the container, and by the front portion of the needle cap and the tubular portion pushed against the seal member, the state that the surface of the seal member faces to the space sealed from the outside is stably maintained.

Furthermore, when the syringe barrel containing the prepared injection solution is taken out to the outside along the barrel axis direction, as the flange portion of the needle cap is pushed against the container side to be clamped by the pushing portion of the holding member, the needle cap fixed at the base end side to the base of the injection needle or the outer surface of the syringe barrel is forcibly separated from the syringe barrel and remains inside of the prefilled syringe kit, and the syringe with the needle cap removed can be taken out to the outside. As a result, it is not necessary to remove the needle cap when using the syringe.

The length of the tubular portion along the tubular axis direction is less than 4 mm.

Because, if it is 4 mm or more, the moving distance required for the injection needle to pass front portion of the needle cap through the inside of the tubular portion to pierce the seal member of the container becomes long, and the operability is deteriorated.

The two-component-mixing prefilled syringe kit according to the present invention is characterized that; it comprises a needle cap which is formed of a material that can be pierced by the injection needle to cover the injection needle from the tip side, and fixed at the base end side to the base of the injection needle or the outer surface of the syringe barrel, and a tubular container adapter which has a tube axis coinciding with the barrel axis direction of the syringe barrel and fixed in close contact with the surface of the seal member at an one end of the tube axis direction, the needle cap has an insertion portion inserted along the tube axis direction of the container adapter from the other end side in the tube axis direction so as to be in close contact with the inner surface of the container adapter at a front portion facing the seal member, the space-forming body is constituted by the container adapter and the insertion portion of the needle cap.

In the present invention, inserting the insertion portion provided at the front portion of the needle cap along the tube axis direction from the other end side in the tube axis direction to the tubular container adapter that one end in the tube axis direction is fixed in close contact with the surface of the seal member, and the insertion portion is in close contact with the inner surface of the container adapter, the space facing the surface of the seal member is formed of a space sealed from the outside. The container and the syringe barrel approach each other, and the tip of the injection needle pierces the expected piercing place in the insertion portion of the needle cap and the seal member at the outlet of the container to communicate the inside of the container with the inside of the syringe barrel, and the liquid component in the container is moved into the syringe barrel to prepare an injection solution. When pulling out the injection needle that has pierced the seal member of the container from the seal member in order to separate the syringe barrel containing the prepared injection solution from the container, the liquid component remaining in the container may leaks to the surface of the seal member, but the liquid component does not leak to the outside because the space facing the surface of the seal member is sealed from the outside by the container adapter and the insertion portion of the needle cap.

In the two-component-mixing prefilled syringe kit according to the present invention, the tubular container adapter has a flange portion being in a direction intersecting with a barrel axis direction of the syringe barrel on an outer side surface of the seal member side, and the holding member has a mount portion for mounting the syringe barrel, and a pushing portion for pushing the flange portion against the container when the syringe barrel mounted on the mount portion is moved along the barrel axis direction to the container side, and a lock part for holding a state where the pushing portion pushes the flange portion against the container.

In the present invention, the holding member with the syringe barrel mounted on the mount portion is moved to the container side along the barrel axis direction, and the tip of the injection needle pierces the insertion portion on the front portion side of the needle cap and the seal member of the container outlet, and the flange portion of the outer side surface of the front portion of the needle cap that intersects the barrel axis direction of the syringe barrel is pushed against the container by the pushing portion provided on the holding member, the state where the pushing portion pushed the flange portion against the container is held by the lock part provided on the holding member. As a result, the tip of the injection needle pierces the insertion portion of the needle cap and the seal member of the container, and by the tubular container adapter fixed in close contact with the surface of the seal member and the insertion portion of the needle inserted so as to be in close contact with the inner surface of the container adapter, the state where the surface of the seal member faces the space sealed from the outside is stably maintained.

Further, when the syringe barrel containing the prepared injection solution is taken out to the outside along the barrel axis direction, as the flange portion of the needle cap is pushed against the container side and clamped by the pushing portion of the holding member, the needle cap fixed at a base end side to the base of the injection needle or the outer surface of the syringe barrel is forcibly separated from the syringe barrel and remains inside of the prefilled syringe kit, and the syringe with the needle cap removed can be taken out to the outside. As a result, it is not necessary to remove the needle cap when using the syringe.

A two-component-mixing prefilled syringe kit according to the present invention is characterized that; it comprises a needle cap which is formed of a material that can be pierced by the injection needle to cover the injection needle from the tip end side, and fixed at the base end side to the base of the injection needle or the outer surface of the syringe barrel; and a container adapter having a tubular portion which is fixed in close contact with the surface of the seal member at one end in the tube axis direction and a flat plate-shape main body portion which covers an opening on the other end side in the tube axis direction of the tubular portion; the main body portion of the container adapter is formed of a material that is pierceable by the injection needle at a portion facing the seal member; and the space-forming body is constituted by the container adapter.

In the present invention, when one end in the tube axis direction of the tubular portion of the container adapter is fixed in close contact with the surface of the seal member, since the opening on the other end side in the tube axis direction of the tubular portion is covered by the flat plate-like main body portion, an internal space of the tubular portion that the surface of the seal member faces is sealed from the outside. The container and the syringe barrel approach each other, the front portion of the needle cap comes in contact with the flat plate-like main body portion of the container adapter, and further, the tip of the injection needle pierces the expected piercing place at the front portion of the needle cap and the seal member at the outlet of the container pierces contacts the front part of the needle cap and a portion facing the seal member in the main body portion of the container adapter, and passing through the inside of the tubular portion of the container adapter to pierce the seal member at the container outlet, after the inside of the container and the inside of the syringe barrel are communicated, the liquid component in the container is moved to the inside of the syringe barrel to prepare the injection solution. When pulling out the injection needle pierced the seal member of the container from the seal member in order to separate the syringe barrel containing the prepared injection solution from the container, the liquid component remaining in the container may leaks to the surface of the seal member, but as the space which the surface of the seal member faces is sealed from the outside by the container adapter, the liquid component does not leak to the outside.

The two-component-mixing prefilled syringe kit according to the present invention is characterized that; the container adapter has a flange portion being in a direction intersecting with a barrel axis direction of the syringe barrel on an outer surface of the seal member side, and the needle cap has a flange portion being in a direction intersecting with a barrel axis direction of the syringe barrel on an outer side surface at the front side, and the holding member has a mount portion for mounting the syringe barrel and a pushing portion for pushing two flange portions of the needle cap and the container adapter against to the container in overlapped state when moving along the barrel axis direction of the syringe barrel to the container side, and a lock part for holding the state where the pushing portion pushes the two flange portions against to the container.

In the present invention, when the holding member with the syringe barrel mounted on the mount portion is moved toward the container along the barrel axis, the tip of the injection needle pierces the front portion of the needle cap, the main body portion of the container adapter and the seal member of the container, the two flange portions of the flange portion on the outside of the front portion the needle cap and the flange portion on the outer side surface of the container adapter are pushed against the container by the pushing portion provided on the holding member, and a state where the pushing portion pushes the two flange portions against the container is held by the lock part provided on the holding member. As a result, the tip of the injection needle pierces the front portion of the needle cap, the main body portion of the container adapter and the seal member of the container, and a state where the surface of the seal member facing the space sealed from the outside is stably maintained by the container adapter fixed in close contact with the surface of the seal member.

Furthermore, when taking out the syringe barrel containing the prepared injection solution to the outside along the barrel axis direction, as the flange portion of the needle cap and the flange portion of the container adapter are pushed against the container side and clamped by the pushing portion of the holding member, the needle cap fixed at base end side to the base side of the injection needle or the outer surface of the syringe barrel is forcibly separated from the syringe barrel and remains inside the prefilled syringe kit, the syringe with the needle cap removed can be taken out. As a result, it is not necessary to remove the needle cap when using the syringe.

The length of the tubular portion along the tubular axis direction is less than 4 mm.

Because, if it is 4 mm or more, the moving distance required for the needle to pass from the needle cap through the inside of the tubular portion of the container adapter and pierces the seal member of the container becomes long, and the operability is deteriorated.

A two-component-mixing prefilled syringe kit according to the present invention is characterized that the lock part is a claw body that can be engaged with a concave portion or a step portion on the outer surface of the container.

In the present invention, since the claw body that is the lock part engages with the concave portion or the step portion of the outer surface of the container, a state that the pushing portion of the holding member pushes the flange portion of the needle cap against the container is maintained by a simple mechanical mechanism.

A two-component-mixing prefilled syringe kit according to the present invention is characterized by comprising an accommodating case that accommodates the whole of the syringe barrel, the needle cap, the container and the holding member in a sealed state to the outside.

Alternatively, a two-component-mixing prefilled syringe kit according to the present invention is characterized by comprising an accommodating case that accommodates the whole of the syringe barrel, the needle cap, the container adapter, the container and the holding member in a sealed state to the outside.
In the present invention, the accommodating case can more effectively prevent the leakage to the outside of the constituents of the injection solution filled in the container and the syringe barrel, including the leakage of the residual liquid in the container.

A two-component-mixing prefilled syringe kit according to the present invention is characterized that the tip of the injection needle is held in the needle cap in a non-penetrated piercing state.

In the present invention, since the tip of the injection needle is held in the needle cap in a non-penetrating state, the tip of the injection needle is located inside the material of the needle cap and is not exposed to the outside. It is possible to store the injection needle and the syringe barrel attached with the injection needle in a stable posture while avoiding the outflow to the outside of the constituent components of the injection solution filled in the syringe barrel.

### Effect of the Invention

According to the two-component-mixing prefilled syringe kit according to the present invention, when an injection solution is prepared by mixing the drug component in the syringe barrel and the liquid component in the container, it is possible to effectively prevent the leakage to the outside of the liquid component remaining in the container after the injection solution is prepared.

### BRIEF DESCRIPTION OF THE DRAWINGS

FIG. 1 is a vertical section of a two-component-mixing prefilled syringe kit according to an embodiment of the present invention.

FIG. 2 is a vertical section of a two-component-mixing prefilled syringe kit according to an embodiment of the present invention.

FIG. 3 is a perspective drawing which shows an external appearance and an internal structure of a needle cap.

FIGS. 4A and 4B are perspective drawings which show structure of a holding member.

FIGS. 5A and 5B are inner surface side perspective drawings of an accommodating case and a dimension explanatory diagram of operating ribs.

FIGS. 6A-6C are a vertical section, a partially enlarged section and a partially assemble drawing of members for making an accommodating case.

FIG. 7 is a sectional arrow-view drawing at VII-VII position of FIG. 1.

FIG. 8 is a sectional arrow-view drawing which shows the position of the accommodating case that rotates against the syringe barrel.

FIGS. 9A-9C are perspective drawings which show an assembly process of the syringe with the holding member.

FIGS. 10A-10D are perspective drawings showing an assembly process of the prefilled syringe kit shown in FIG. 1.

FIGS. 11A-11C are sections explaining the preparation operation of the injection solution by the prefilled syringe kit of FIG. 1.

FIGS. 12A and 12B are a partially enlarged vertical section of FIG. 11C and a dimension explanatory drawing of the tubular portion at the tip of the needle cap.

FIGS. 13A-13C are perspective drawings showing a process for preparing an injection solution by the prefilled syringe kit of FIG. 1.

FIGS. 14D-14F are perspective drawings showing a process for preparing an injection solution by the prefilled syringe kit of FIG. 1.

FIGS. 15G and 15H are perspective drawings showing take-out of syringe from prefilled syringe kit of FIG. 1.

FIGS. 16A-16C are perspective drawings showing a disposal preparation process of a used syringe.

FIG. 17 is a schematic section enlarging a part of the accommodating case after the disposal preparation process is completed.

FIG. 18 is a partially enlarged section of the prefilled syringe kit according to a 1st another embodiment of present invention.

FIGS. 19A-19D are perspective drawings showing an assembly process of the syringe with the holding member.

FIGS. 20A-20C are a perspective drawing showing an assembly process of the prefilled syringe kit shown in FIG. 18.

FIGS. 21A and 21B are sections explaining the injection solution preparing operation by the prefilled syringe kit of FIG. 18.

FIG. 22 is a partially enlarged section of the prefilled syringe kit according to a 2nd another embodiment of present invention.

FIGS. 23A and 23B are sections explaining the injection solution preparing operation by the prefilled syringe kit of FIG. 22.

FIG. 24 is a partially enlarged vertical section of a prefilled syringe kit according to a third another embodiment of the present invention.

FIGS. 25A-25D are perspective drawings showing an assembly process of the prefilled syringe kit shown in FIG. 24.

### MODE FOR CARRYING OUT THE INVENTION

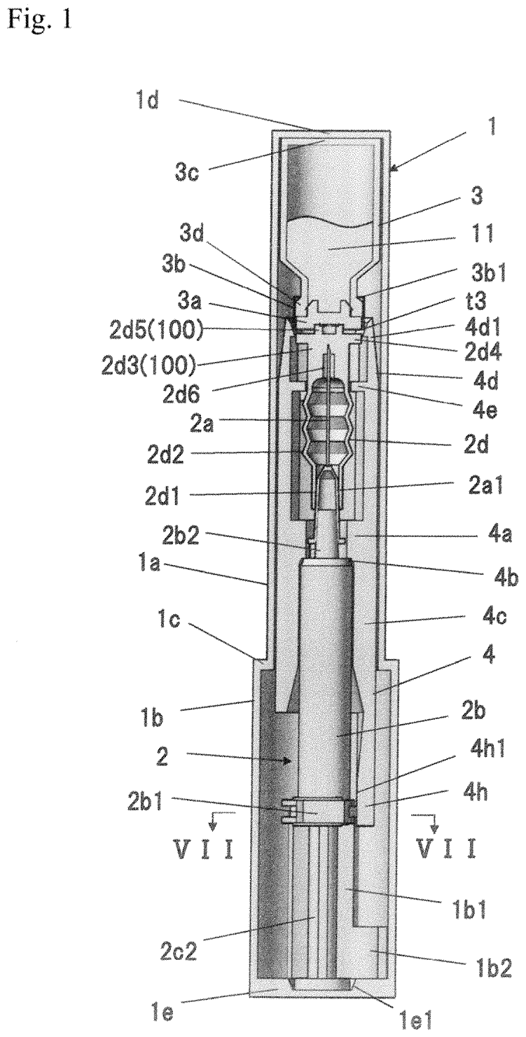

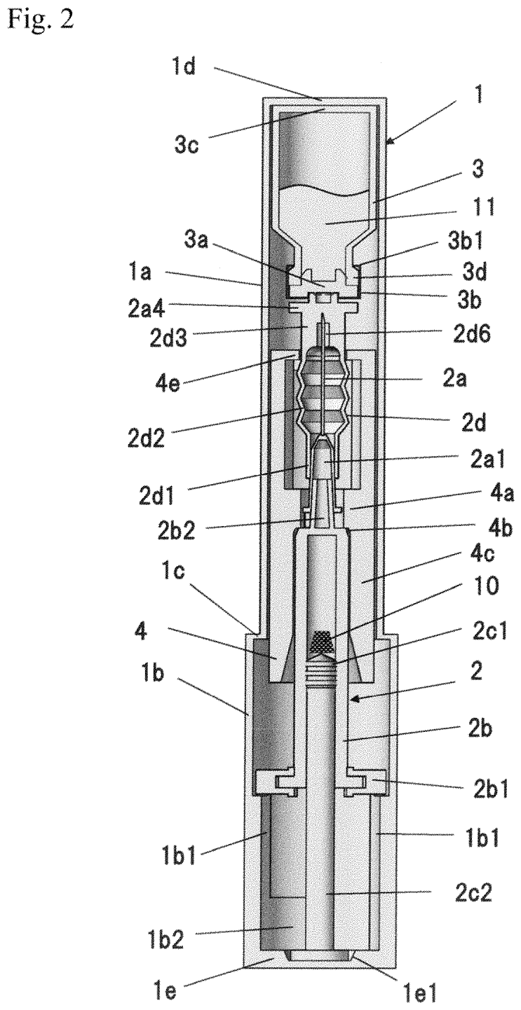

Hereinafter, embodiments of a two-component-mixing prefilled syringe kit according to the present invention will be described with reference to the drawings. FIG. 1 and FIG. 2 are vertical sections of a two-component-mixing prefilled syringe kit according to the present invention. Also, FIG. 1 and FIG. 2 show vertical sections at positions where they are rotationally symmetrical by 90 degrees to each other in a cross-section of the prefilled syringe kit.

A prefilled syringe kit shown in FIG. 1 and FIG. 2 includes a cylindrical accommodating case 1 which is elongated in an axial direction. The accommodating case 1 has a structure that in a state where a small-diameter cylindrical portion 1a having a small outer diameter and a large-diameter cylindrical portion 1b having a large outer diameter are aligned with their axes each other, the outer circumference of one end of the small-diameter cylindrical portion 1a and the inner circumference of one end of the large-diameter cylindrical portion 1b are connected by an toroidal thin plate portion 1c. The other ends of the cylindrical portions 1a and 1b are respectively closed by walls 1d and 1e. As a result, the inside of the accommodating case 1 is sealed from the outside. The accommodating case 1 is made of a transparent hard vinyl chloride resin or the like.

Inside the accommodating case 1, the syringe 2, the holding member 4 and the container 3 are sequentially arranged along the axial direction of the accommodating case 1. The holding member 4 holds the syringe 2 to position the syringe 2 with respect to the container 3. Inside the small-diameter cylindrical portion 1a, the container 3, most of the holding member 4 and the front-side part of the syringe 2 are located, and inside the large-diameter cylindrical portion 1b, a part of the holding member 4 and the rear-side part of the syringe 2 are located.

The syringe 2 comprises a syringe barrel 2b, an injection needle 2a attached to a front end portion 2b2 of the syringe barrel 2b, a plunger (also referred to as a gasket) 2c1 inserted into a rear end opening of the syringe barrel 2b, and the driving rod 2c2 connected a rear portion of the plunger 2c1. The injection needle 2a is fixed with the flange portion 2a1 of the base side pushed into the tip end side of the front end portion 2b2 of the syringe barrel 2b. The syringe barrel 2b is made of glass such as borosilicate glass. The plunger 2c1 is generally made of butyl rubber, and is coated with silicone oil in order to smoothly slide on the inner surface of the syringe barrel 2b. As a result, the inner surface of the syringe barrel 2b that contacts the plunger 2c1 is also coated with silicone oil. Also, the silicone oil coating method is generally a method that a gasket is put into water dispersing silicone oil and stirred.

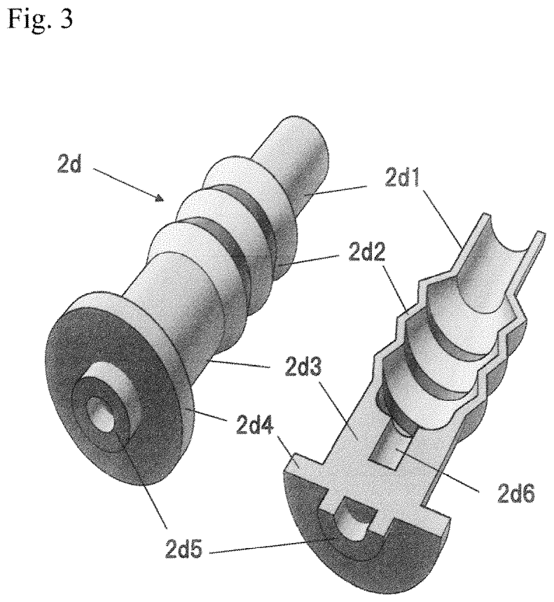

A needle cap 2d which cover the injection needle 2a from the tip side is provided. FIG. 3 is a perspective drawing showing the appearance and internal structure of the needle cap. The needle cap 2d has a structure that a cylindrical portion 2d1 on the base end side, a bellows portion 2d2 having an outer diameter larger than that of the cylindrical portion 2d1, and a cylindrical front portion 2d3 having an outer diameter intermediate between the outer diameter of the cylindrical portion 2d1 and the outer diameter of the bellows portion 2d2 continue sequentially coaxially along the axial direction of the cylindrical portion 2d1. The front portion 2d3 is provided with a disc-shaped flange portion 2d4 on the side surface of the tip end portion, and is provided with a cylindrical tubular portion 2d5 having a diameter smaller than the outer diameter of the front portion 2d3 on the front surface of the tip end portion. The tip of the tubular portion 2d5 is located on one plane orthogonal to the axial direction of the syringe barrel 2b. Further, a cylindrical cavity 2d6 is formed in the front portion 2d3 from an end on the side of the bellows portion 2d2 to an intermediate position in the axial direction, and the cavity 2d6 communicates with the inner space of the cylindrical portion 2d1 and the bellows portion 2d2. The needle cap 2d is made of butyl rubber, for example. The tip of the injection needle 2a is pierced through the cavity 2d6 into the tip side portion of the front portion 2d3 in a non-penetrating state. Also, the open end of the cylindrical portion 2d1 is fitted and held on the outer circumference of the flange portion 2a1 of the injection needle 2a.

The container 3 is a cylindrical container having an outer diameter slightly smaller than the inner diameter of the small-diameter cylindrical portion 1a of the accommodating case 1, and is made of glass such as borosilicate glass. The container 3 is specifically a vial. The outlet of the container 3 is sealed by a seal member 3a. The seal member 3a is fixed by a stopper 3b so as to close the outlet opening of the container 3. The stopper 3b includes a circular plate portion having a central part opening, and a cylindrical base portion that is continuous with the circumferential edge of the circular plate portion and extends toward the bottom side of the container 3. The circular plate portion of the stopper 3b pushes the seal member 3a against the outlet of the container 3. The cylindrical base portion of the stopper 3b is narrowed inward so that the tip side surrounds the flange portion 3d on the outlet side of the container 3 to form a step portion 3b1. The seal member 3a is made of butyl rubber or chlorinated butyl rubber, and the stopper 3b is made of metal or hard plastic.

a powder drug 10 is filled in the inside of the syringe barrel 2b as a first component constituting the injection solution. The powder drug is crystalline powder, freeze-dried powder, or the like. On the other hand, in the inside of the container 3, a solution 11 for dissolving the powder drug 10 or a dispersion liquid 11 for dispersing the powder drug 10 is filled as a second component constituting the injection solution. In addition, both the first component and the second component may include multiple types of substances other than one type of substance.

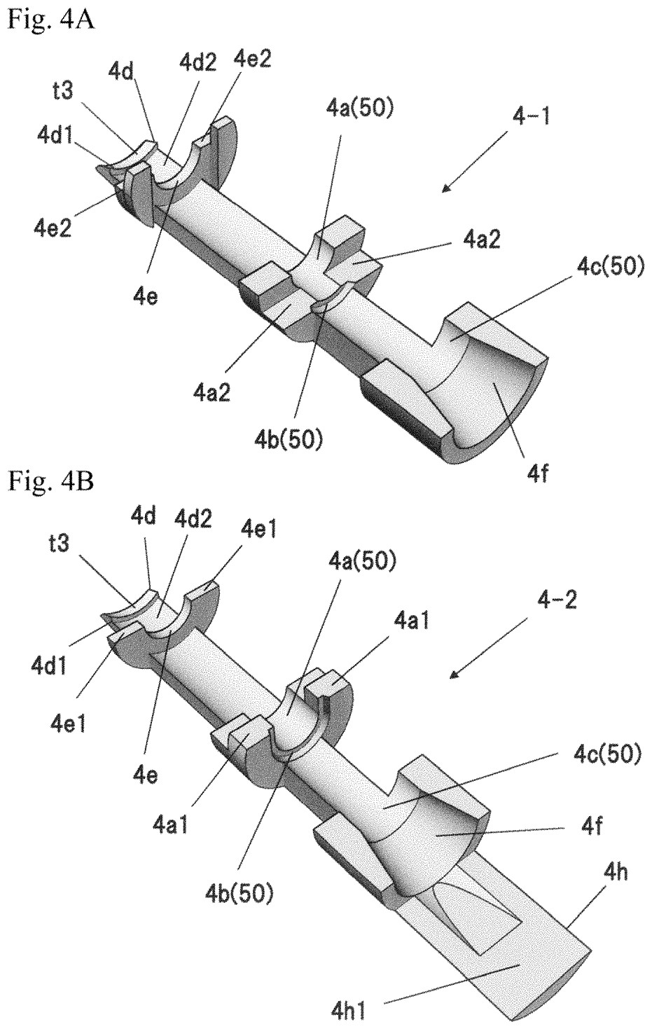

Next, the holding member 4 is described. FIGS. 4A and 4B is a perspective drawing showing the structure of the holding member 4.

The holding member 4 is a cylindrical member having an outer diameter slightly smaller than the inner diameter of the small-diameter cylindrical portion 1a of the accommodating case 1, and is constituted by two semi-cylindrical members 4-1, 4-2 which are vertically divided by a plane passing through the axis (FIGS. 4A, 4B). The syringe 2 is housed inside the holding member 4 with the barrel axes aligned. The holding member 4 is made of a transparent hard vinyl chloride resin or the like.

The holding member 4 is provided with a toroidal rib 4a which holds the flange-shaped toroidal part at the rear end of the flange portion 2a1 of the base side of the injection needle 2a, and a toroidal step portion 4b which continues to the rear side of the toroidal rib 4a, being in contact with the circumferential edge corner portion to the front side of the syringe barrel 2b to regulate the position, and a cylindrical guide 4c which has an inner diameter slightly larger than the outer diameter of the syringe barrel 2b, to regulate the position in the radial direction of the syringe barrel 2b on the rear side of the toroidal step portion 4b. That is, the toroidal rib 4a, the toroidal step portion 4b, and the cylindrical guide 4c constitute the mount portion 50 provided in the holding member 4. At the rear end portion of the holding member 4, a cylindrical guide 4f having a diameter increased toward the rear end side is provided to guide the insertion of the used syringe 2. Further, on one of the semi-cylindrical members 4-2 (FIG. 4B), a rotation stop portion 4h having a crescent section is extended from the end of the cylindrical guide 4f in the barrel axial direction. The rotation stop portion 4h has a flat-face inner circumferential surface 4h1 parallel to the barrel axis direction.

A movable piece 4d is provided at the front end of the holding member 4, and a toroidal plate-shaped pushing portion 4e protruding inward is provided at a base side place of the movable piece 4d. The movable piece 4d includes a hook portion 4d1 at the tip and a support portion 4d2 which supports the hook portion 4d1 and is elastically bendable in the radial direction. The movable pieces 4d are provided at two locations of 180-degree rotational symmetric position in a cross section orthogonal to the barrel axis. The hook portion 4d1 is formed with an inclined surface t3 that is inclined so as to form an acute angle with respect to the barrel axis center direction and has a surface direction facing the barrel axis center side.

In one semi-cylindrical member 4-1, the pushing portion 4e is formed by a half circumference, and a pair of step portions 4e2, 4e2 are formed at 180-degree rotational symmetry positions by parts which are respectively axially adjacent and radially protrude from both circumferential edges of the pushing portion 4e. In the other semi-cylindrical member 4-2, the pushing portion 4e is formed by a half circumference, and a pair of convex portions 4e1, 4e1 protruding in the circumferential direction are formed at both edges. Further, in the one semi-cylindrical member 4-1, an axial rear portion of the toroidal rib 4a is formed shorter than a half circumference, and a pair of step portions 4a2, 4a2 having low height are formed. In the other semi-cylindrical member 4-2, the rear half in the axial direction of the toroidal rib 4a is formed longer than the half circumference, and a pair of step portions 4a1, 4a1 having high height are formed. Then, the semi-cylindrical member 4-1 shown in FIG. 4A is turned upside down (turned over) and overlapped on the semi-cylindrical member 4-2 shown in FIG. 4B, inset each of the pair of convex portions 4e1, 4e1 into each of the pair of the step portions 4e2, 4e2 at the location of the pushing portion 4e, and hitting each of the pair of step portions 4a1, 4a1 against each of the pair of the step portions 4a2, 4a2 at the location of the toroidal rib 4a to fit together, so that both of the semi-cylindrical members 4-1, 4-2 are joined together to form a cylindrical holding member 4.

As shown in FIG. 1 and FIG. 2, the bottom portion 3c on the side opposite to the seal member 3a of the container 3 is in contact with the wall 1d of the accommodating case 1, so that the axial position of the container 3 is regulated. Also, the inclined surface t3 of the hook portion 4d1 on the tip of the movable piece 4d of the holding member 4 is in contact with the circumferential edge corner portion of the stopper 3b of the container 3. As a result, the syringe 2 is positioned with respect to the container 3 in a state where the tip of the injection needle 2a pierced the needle cap 2d in a non-penetrate state faces the seal member 3a of the container 3 via the needle cap 2d.

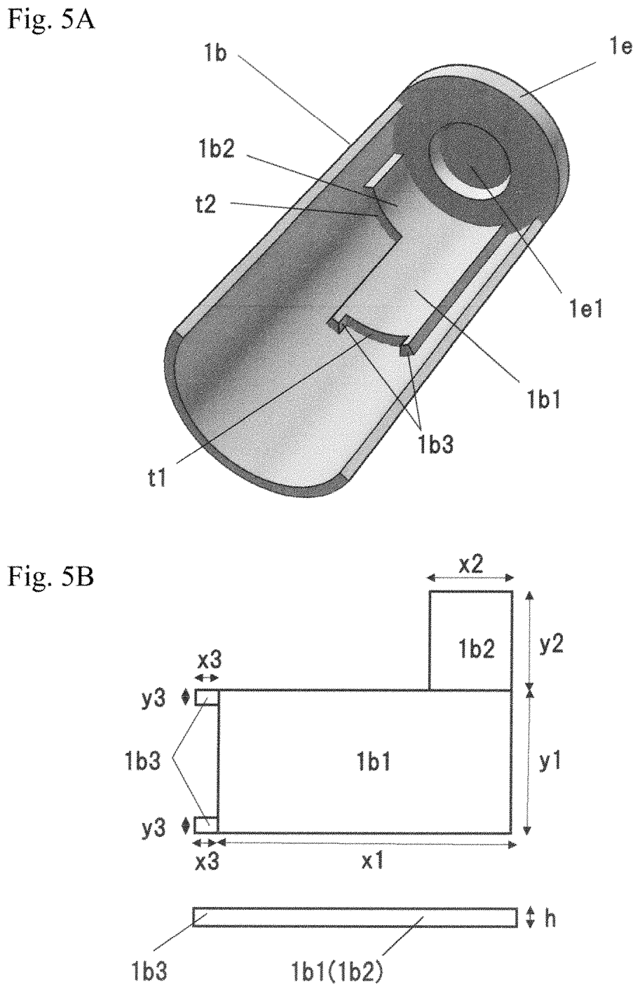

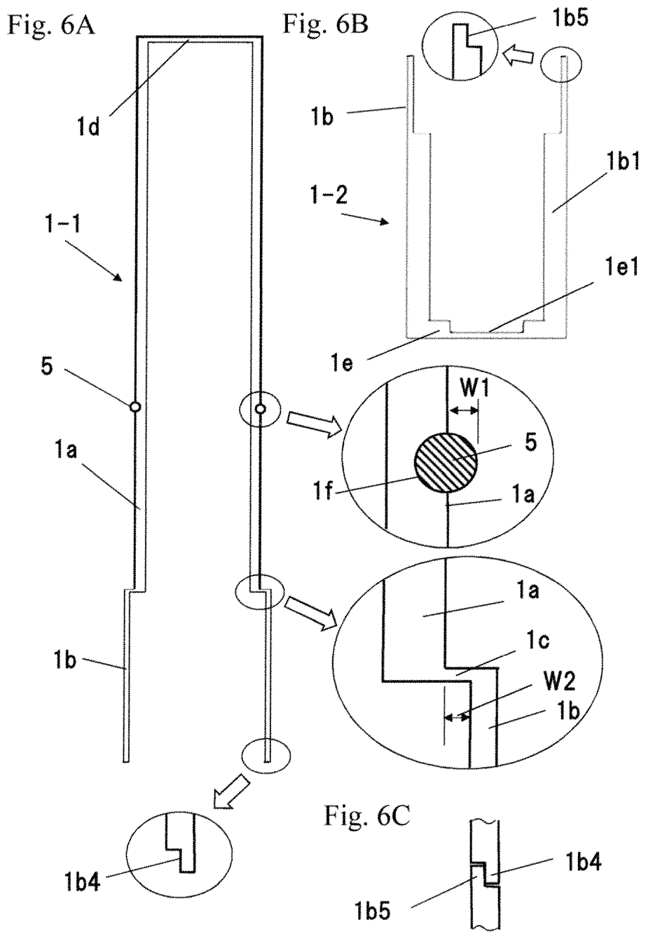

A structure of the accommodating case 1 is described by FIGS. 5A, 5B, and FIGS. 6A-6C. FIGS. 5A and 5B are perspective drawings of the inner-surface side of the accommodating case and an explanatory drawing of dimensions of the operating ribs, and FIGS. 6A-6C are vertical sections and a partially enlarged section and a partially assemble drawing of members for manufacturing the accommodating case. FIG. 5A shows the inner surface side of the accommodating case 1 in which the large-diameter cylindrical portion 1b is virtually split vertically along the axial direction, and a half of the barrel is cut away except for the wall 1e. FIG. 5B shows the dimensions of the ribs developed in the circumferential direction.

A rib 1b1 and a second rib 1b2 protruding from the inner circumferential wall at a uniform height h are provided on the inner surface side of the large-diameter cylindrical portion 1b. The rib 1b1 is a step portion of a height h having a length x1 from the wall 1e and protruding in a rectangular shape with a circumferential width y1. The second rib 1b2 is a step portion of a height h which is adjacent to the rib 1b1 in the circumferential direction, and has a length x2 from the wall 1e that is shorter than the length x1 of the rib 1b1 (x1>x2), and protruding in a rectangular shape with a circumferential width y2. The rib 1b1 has a side surface t1 perpendicular to the barrel axis direction on the side opposite to the wall 1e. The second rib 1b2 has a side surface t2 perpendicular to the barrel axis direction on the side opposite to the wall 1e. A pair of third ribs 1b3 having a length x3 in the axial direction, a width y3 in the circumferential direction, and a height h is provided at both edges in the circumferential direction of the side surface t1 of the rib 1b1. The third rib 1b3 is elongated in the barrel axis direction, and the length x3 in the barrel axis direction is set to be approximately the same as the thickness of the flange portion 2b1 at the rear edge of the syringe barrel 2b. The ribs 1b1, the second ribs 1b2, and the third ribs 1b3 are provided in pairs at positions 180 degrees rotationally symmetrical in the cross section of the accommodating case 1, respectively.

FIG. 6A shows a case component 1-1 having a small-diameter cylindrical portion 1a and a part of the large-diameter cylindrical portion 1b connected to the small-diameter cylindrical portion 1a by a thin plate portion 1c. In the case component 1-1, the end on the side of the large-diameter cylindrical portion 1b is open, and the end on the side of the small-diameter cylindrical portion 1a becomes the wall 1d. FIG. 6B shows a case component 1-2 constituting the remaining part of the large-diameter cylindrical portion 1b. In the case component 1-2, one end in the axial direction is open and the other end becomes the wall 1e.

A concave groove 1f having a semicircular section is formed on the outer circumferential location of the small-diameter cylindrical portion 1a over the entire of circumferential direction thereof, and an O-ring 5 is fitted in the concave groove 1f. The protrusion amount W1 of the O-ring 5 from the outer circumferential surface of the small-diameter cylindrical portion 1a is arranged larger other than the difference of positions in the radial direction between the outer circumferential surface of the small-diameter cylindrical portion 1a and the inner circumferential surface of the large-diameter cylindrical portion 1b (that is, a width of the toroidal thin plate portion 1c) W2 (W1>W2).

A step portion 1b4 of a shape in which a thick outer portion of the large-diameter cylindrical portion 1b protrudes in the axial direction over the entire circumference is formed at an open-side end of the case component 1-1. On the other hand, a step portion 1b5 of a shape in which a thick inner portion of the large-diameter cylindrical portion 1b protrudes in the axial direction over the entire circumference is formed at an open-side end of the case component 1-2.

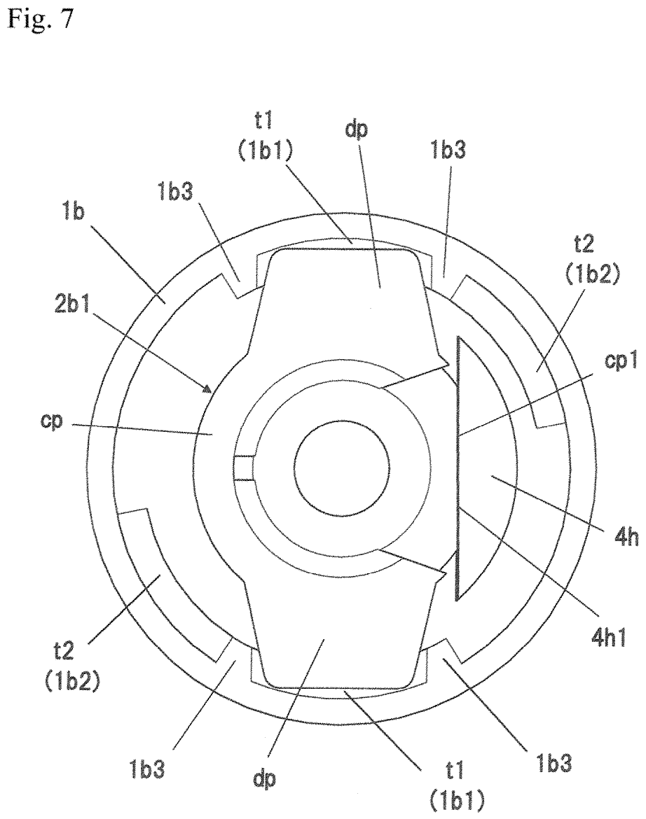

Next, based on FIG. 1 and FIG. 7, the positioning and holding structure of the rear side of the syringe 2 by the accommodating case 1 is described. FIG. 7 is a section arrow-view drawing at the position VII-VII in FIG. 1

The flange portion 2b1 at the rear end of the syringe barrel 2b has an outer shape in which trapezoidal portions dp narrowing outward are connected to both sides that are rotationally symmetrical by 180 degrees of the circular portion cp in the axial direction view. The circular portion cp has a straight outer circumferential portion cp1 on one end side in the direction orthogonal to the direction connecting the two trapezoidal portions dp. The tip of each trapezoidal portion dp of the flange portion 2b1 is in contact with the side surface t1 of the rib 1b1 between the pair of third ribs 1b3. As a result, the syringe barrel 2b is positioned in the axial direction, and when the syringe barrel 2b rotates around the axis center, it is regulated that each of the trapezoidal portions dp of the flange portion 2b1 comes outside further other than the third ribs 1b3 by the third ribs 1b3 on both sides, and the flange portion 2b1 is held in a state of being in contact with the side surface t1 of the rib 1b1. Also, the inner circumferential surface 4h1 of the rotation stop portion 4h of the holding member 4 is in contact with the straight outer circumferential portion cp1 of the flange portion 2b1, whereby the syringe barrel 2b and the holding member 4 rotate integrally around the axis center.

A circular concave portion 1e1 having a constant depth is formed in the center of the inner surface side of the wall 1e. The diameter of the concave portion 1e1 is larger than the outer shape of the flange at the rear end of the driving rod 2c2, and the depth of the concave portion 1e1 is approximately the same as the thickness of the flange at the rear end of the driving rod 2c2. The flange at the rear end of the driving rod 2c2 is in contact with the bottom of the concave portion 1e1 and the axial position of the driving rod 2c2 and thus the plunger 2c1 is regulated.

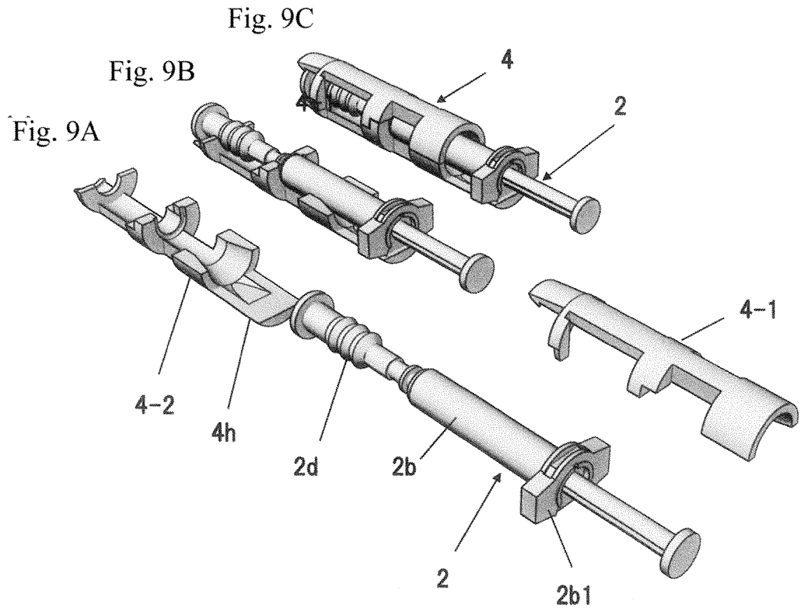

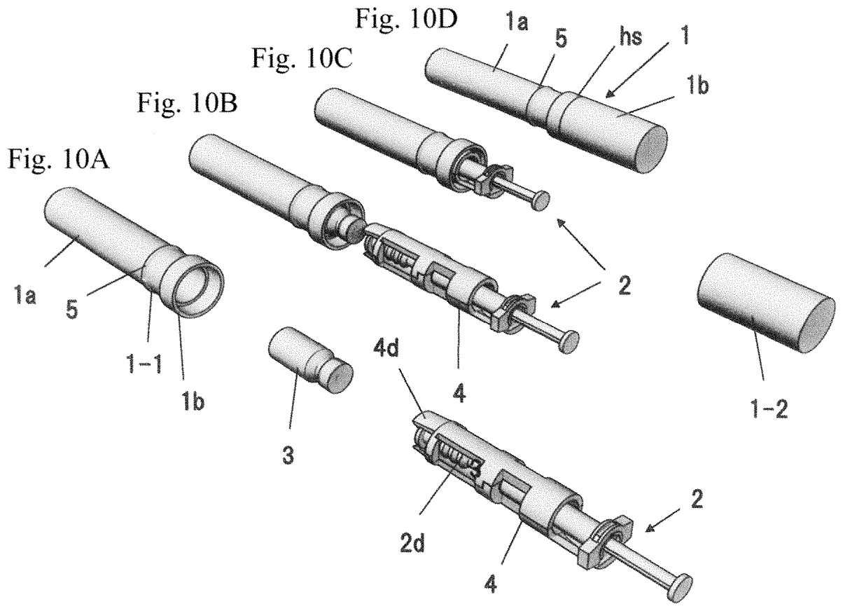

Next, the assembly of the prefilled syringe kit is described. FIGS. 9A-9C are perspective drawings showing an assembling process of the syringe to the holding member, and FIGS. 10A-10D are perspective drawings showing an assembling process of the prefilled syringe kit.

In the assembling process of the syringe 2 to the holding member 4, first, the syringe 2 pre-filled with the powder drug 10 and having the needle cap 2d attached thereto is set from above to the semi-cylindrical member 4-2 facing upward with the inside (FIGS. 9A, 9B). At this time, the position is set so that the straight outer circumferential portion cp1 of the flange portion 2b1 comes in contact with the rotation stop portion 4h of the holding member 4 (see FIG. 7). Next, the semi-cylindrical member 4-1 with the inside facing downward is put over the syringe 2 and integrally connected with the semi-cylindrical member 4-2 (FIGS. 9B, 9C). As a result, the holding member 4 is formed by the semi-cylindrical members 4-1, 4-2 that are integrally connected, and the syringe 2 is assembled to the holding member 4 in a state where the syringe barrel 2b is fixed and supported by the holding member 4.

In the assembling process of the prefilled syringe kit, first, the container 3 is inserted from the bottom side into the end opening of the large-diameter cylindrical portion 1b side of the case component 1-1 (FIG. 10A). Subsequently, the holding member 4 in which the syringe 2 with the needle cap 2d is set is inserted to the inside of the case component 1-1 with the movable piece 4d facing forward (FIGS. 10A, 10B, 10C). Next, the case component 1-2 is covered so as to cover the rear side of the syringe 2 popping out of the end opening of the case component 1-1, and the opening ends of both case components 1-1, 1-2 are connected so that the both step portions 1b4, 1b5 are fitted each other (FIGS. 10C, 10D and FIG. 6C). At this time, the cylindrical tubular portion 2d5 of the tip of the needle cap 2d becomes into a state of being close to and facing the seal member 3a of the container 3. Further, as shown in FIG. 7, in a state where the flange portion 2b1 of the syringe barrel 2b is in contact with the rib 1b1, the two trapezoidal portions dp on both sides are held between the pair of third ribs 1b3 to regulate the rotation position.

Finally, the step portions 1b4, 1b5 of both the joined case components 1-1, 1-2 are thermally joined to form a joint portion hs (FIG. 10D), and the sealed case 1 is constituted. The thermal joining is, for example, an electric iron method, in this method while pushing the iron heated to high temperature on the joint part of the case components 1-1, 1-2, the accommodating case 1 is rotated with respect to the fixed iron (or, the iron is rotated with respect to the fixed accommodating case 1), so that the material (vinyl chloride resin) of the accommodating case 1 is welded. Further, it is possible to use a method in which a high temperature hot air is blown onto the joint part of the case components 1-1, 1-2 to weld them together.

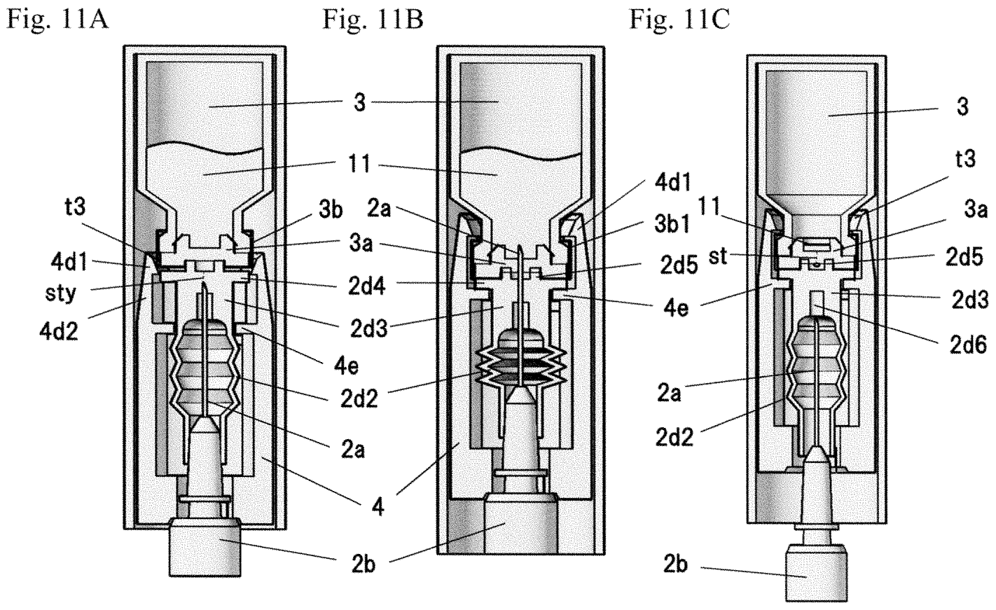

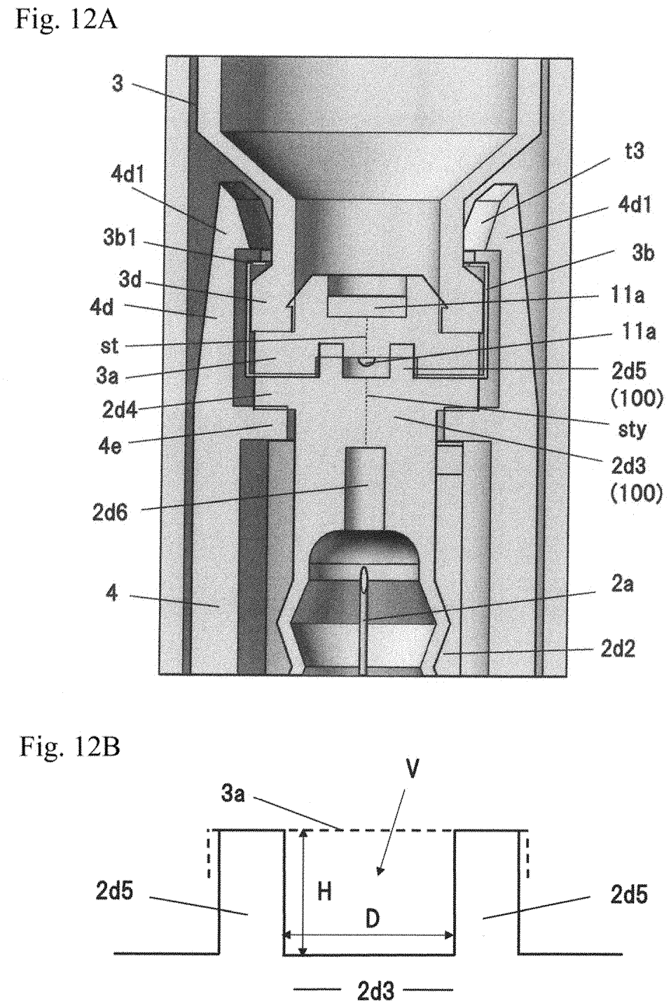

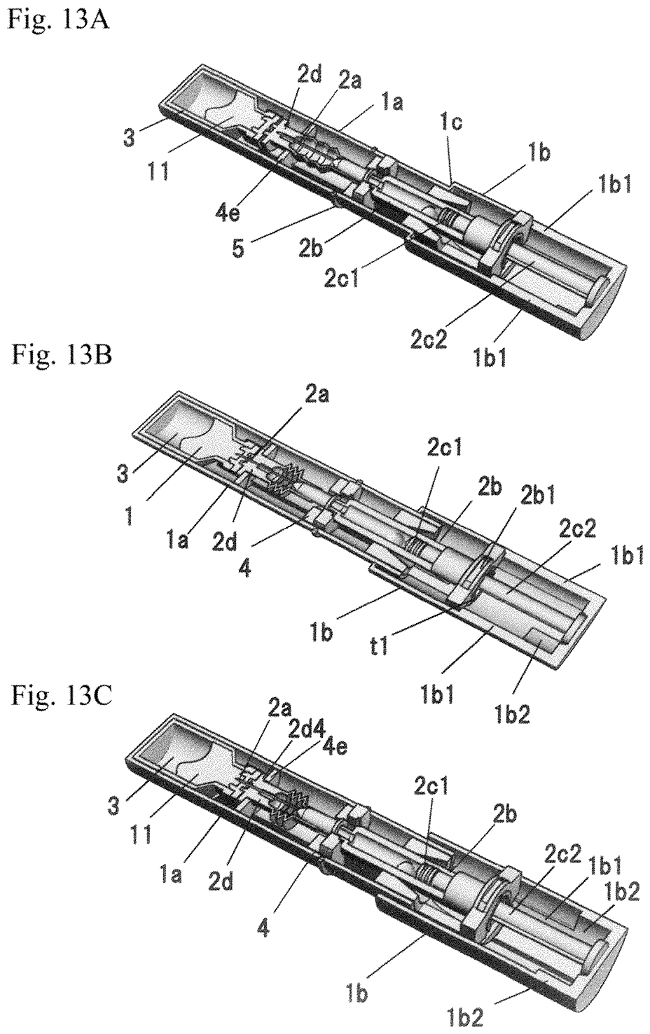

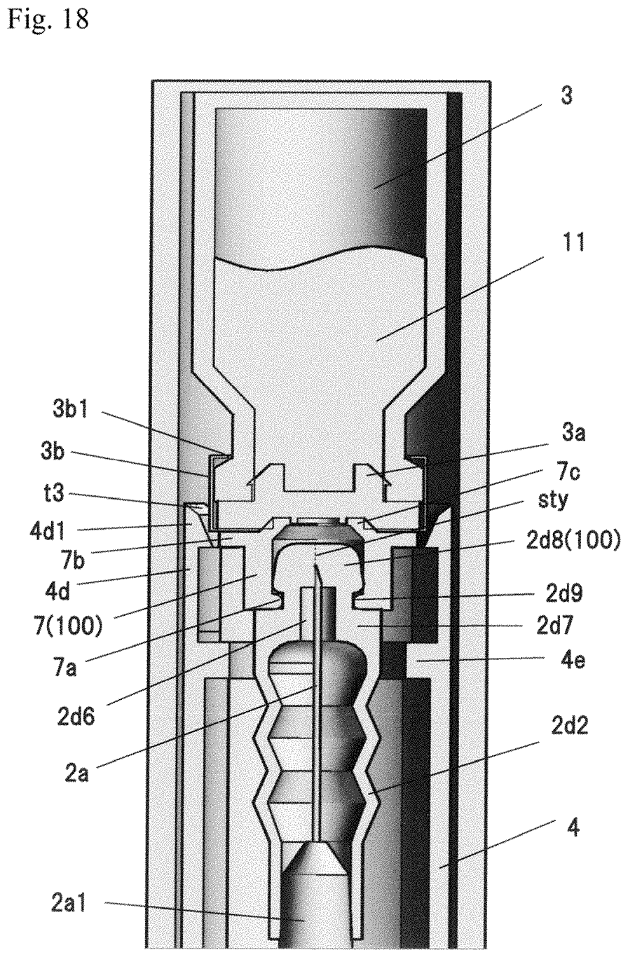

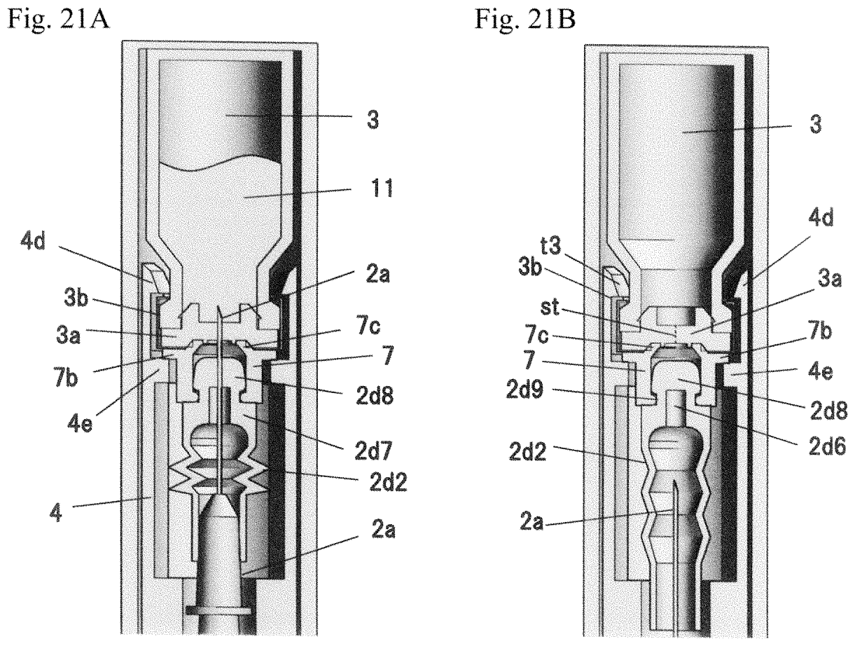

Next, an injection solution preparing operation by the two-component-mixing prefilled syringe kit according to the present invention is described. FIGS. 11A-11C are sections for explaining the injection solution preparing operation by the prefilled syringe kit of FIG. 1, and FIGS. 12A, 12B are a partially enlarged section of FIGS. 11C and a dimension explanatory drawing of the tubular portion at the tip of the needle cap. Note, in FIGS. 11A to 12B (the same applies to FIG. 13A to FIG. 15H, FIG. 18, and FIG. 21A to FIG. 23B), for convenience of explanation, the accommodating case 1 is virtually vertical divided along the axial direction and a half of the cylindrical portion is removed, and it is drawing so that the inside is visible.

When the syringe barrel 2b is pushed into the container 3 side, the bellows portion 2d2 of the needle cap 2d contracts, and the tip of the injection needle 2a that has been pierced to the midway point of the front portion 2d3 of the needle cap 2d penetrates the expected piercing place sty and the seal member 3a to project inside the container 3, and the tubular portion 2d5 of the front portion 2d3 of the needle cap 2d is pushed against the seal member 3a (FIGS. 11A, 11B). At this time, the holding member 4 is also pushed by the syringe barrel 2b to move to the container 3 side, and the hook portion 4d1 of the movable piece 4d which is in contact with the circumferential edge corner portion of the stopper 3b of the container 3 at the inclined surface t3 slides to move outward, and the supporting portion 4d2 bends outward, and the hook portion 4d1 that has moved outward passes by sliding on the side surface of the stopper 3b. At the same time, the pushing portion 4e of the holding member 4 pushes the flange portion 2d4 of the needle cap 2d against the front surface of the stopper 3b of the container 3. Then, the hook portion 4d1 that has slid outward returns to the inside and is hanged by the step portion 3b1 on the base side of the stopper 3b, and the holding member 4 is mechanically connected to the container 3 at this position. As a result, the state where the tip of the injection needle 2a projects into the container 3 and the state where the tubular portion 2d5 of the front portion 2d3 of the needle cap 2d is pushed against the seal member 3a are maintained (locked). That is, the hook portion 4d1 provided on the movable piece 4d corresponds to the lock part which locks the state where the pushing portion 4e is hit against the container 3 and the claw body which can be fitted with the step portion 3b1 on the outer surface of the container 3.

Next, the driving rod 2c2 is operated with a finger to push the plunger 2c1, after the air in the syringe barrel 2b is sent into the container 3 to increase the pressure in the container 3, the finger is released from the driving rod 2c2 and the plunger 2c1 is made free, then the liquid 11 in the container 3 is sucked to inside of the syringe barrel 2b through the injection needle 2a by the internal pressure, and the plunger 2c1 is returned to the original position. The liquid 11 sucked in the syringe barrel 2b is mixed with the powder drug 10 inside the syringe barrel 2b to prepare the injection solution. Finally, the syringe barrel 2b is gripped with the fingers, the injection needle 2a is pulled out from the container 3, and is pulled back to the inside of the needle cap 2d (FIG. 11C, FIG. 12A). At this time, a liquid 11a being a part of the liquid 11 remaining in the container 3 may overflow onto the surface of the seal member 3a through the needle piercing place st of the seal member 3a, but, since the space surrounded by the front portion 2d3 of the needle cap 2d pushed against the seal member 3a and the tubular portion 2d5 and the seal member 3a is sealed from the outside, the liquid 11a overflowing from the container 3 does not leak to the outside. That is, the space-forming body 100 is constituted by the front portion 2d3 of the needle cap 2d and the tubular portion 2d5.

Next, a space V (closed space) surrounded by the surface of the seal member 3a and the front portion 2d3 and the tubular portion 2d5 of the needle cap 2d is described by FIG. 12B. Here, it is assumed that the tubular portion 2d5 is cylindrical. D is the inner diameter of the tubular portion 2d5, and H is the height (length along the tubular axis direction) of the tubular portion 2d5. Note, the tubular portion 2d5 pushed against the seal member 3a is compressed in the pushing direction and is slightly deformed, but the effect of this deformation is not taken into consideration in the description. Generally, the seal member 3a of the vial 3 has a circular shape in a plane view, and the size (diameter) of the area assigned to the needle piercing part of the seal member 3a is about 7 mm. When the tubular portion 2d5 is pushed into the area of the needle piercing part, assuming the thickness of the circumferential wall of the tubular portion 2d5 is 1 mm, the inner diameter D of the tubular portion 2d5 is about 4 mm at maximum. The height H of the tubular portion 2d5 cannot be increased so much in consideration of downsizing of the device and manufacturing. For example, when the height H is 1 mm, the volume of the space V is 0.0125 ml when the inner diameter D is 4 mm, and the volume of the space V is 0.00314 ml when the inner diameter D is 2 mm. Also, when the height H is increased to 3 mm and the inner diameter D is decreased to 3 mm, the volume of the space V is 0.0212 ml. The volume of the space V is calculated by the formula V=π(D/2)2H.

After filling 2 ml of purified water (hereinafter referred to as a liquid) in a vial 3 having an inner space of 4 ml, the vial 3 is turned upside down so that the seal member 3a is located on the lower side, and when the syringe 2 having the needle tip 27G is pierced to the seal member 3a and 2 ml of air is injected into the vial 3, a liquid leakage from the seal member 3a is confirmed. Therefore, in a state that the tubular portion 2d5 at the tip of the needle cap 2d is pushed and set against the surface of the seal member 3a, the syringe 2 with the needle tip 27 G is pierced into the front portion 2d3 of the needle cap 2d and is pierced the seal member 3a passed through the inside of the tubular portion 2d5, and injecting 2 ml of air in a vial 3 filled with 2 ml of liquid. Table 1 below shows the test result of liquid leakage to the outside, needle movement distance (operability), compactness, and comprehensive evaluation. As experimental conditions, the inner diameter D was changed to 1.0 and 2.0 mm for the height H=1.0 mm of the tubular portion 2d5, and the inner diameter D was changed to 2.0 and 3.0 mm for the height H=2.0 mm of the tubular portion 2d5, and the inner diameter D was changed 1.0, 2.0, 3.0 and 4.0 mm for the height H=3.0 mm and 4.0 mm each of the tubular portion 2d5.

TABLE 1
Syringe condition: Needle tip 27G
Vial conditions: Inner space 3 ml Filled liquid amount 2 ml Injected air amount 2 ml

| | | | | Needle | | |
|---|---|---|---|---|---|---|
| Needle cap conditions | | | Liquid leak | movement | | Comprehensive |
| H(mm) | D(mm) | V(ml) | status | distance | Compactness | evaluation |
| 1.0 | 1.0 | 0.0008 | X | ◯ | | |
| ″ | 2.0 | 0.0031 | Δ | ◯ | | |
| 2.0 | 2.0 | 0.0062 | Δ | ◯ | ◯ | |
| ″ | 3.0 | 0.0141 | ◯ | ◯ | ◯ | ⊚ |
| 3.0 | 1.0 | 0.0023 | X | Δ | ◯ | (Posture instability) |
| ″ | 2.0 | 0.0094 | ◯ | Δ | ◯ | ⊚ |
| ″ | 3.0 | 0.0212 | ◯ | Δ | ◯ | ⊚ |
| ″ | 4.0 | 0.0377 | ◯ | Δ | Δ | |
| 4.0 | 1.0 | 0.0031 | Δ | X | Δ | |
| ″ | 2.0 | 0.0126 | ◯ | X | Δ | |
| ″ | 3.0 | 0.0283 | ◯ | X | Δ | |
| ″ | 4.0 | 0.0502 | ◯ | X | Δ | |

| | | | | |
|---|---|---|---|---|
| Evaluation criteria | X: Blow out | X: Long | Δ: a little large | A good balance of |
| | Δ: Leak a little | Δ: a little long | ◯: No problem | needle travel |
| | ◯: No leakage | ◯: No problem | | distance (operability) |
| | | | | and compactness |
| | | | | without liquid leakage. |

Regarding liquid leakage, there is no liquid leakage when the volume of the space V is larger than 0.0094 ml. The needle movement distance is a distance required to pierce to the vial from the needle cap with the needle, the allowable range is less than 4 mm since the operability deteriorates when the height H of the tubular portion 2d5 is 4 mm or more. Compactness decreases as the size of the tubular portion 2d5 increases. Evaluating comprehensively that the needle movement distance (operability) and compactness are met well-balanced without liquid leakage, three conditions of an inner diameter D 3.0 mm at height H 2.0 mm of the tubular portion 2d5, an inner diameter D 2.0 at height H 3.0 mm of the tubular portion 2d5, and an inner diameter D 3.0 mm at height H 3.0 of the tubular portion 2d5 were set as preferable conditions. Also, if the ratio of the height H to the inner diameter D is large, the posture when the tubular portion 2d5 is pushed against the surface of the seal member 3a may become unstable (for example, when the inner diameter D 1.0 mm at the height H 3.0 mm of the tubular portion 2d5). The volume of the space V is allowable in the range of 0.001 ml to 0.03 ml, and more preferably in the range of 0.005 ml to 0.025 ml. The inner diameter D is allowable in the range of 1.0 to 5.0 mm, and the more preferable range is 2.0 mm to 4.0 mm.

Note, the tubular portion 2d5 is not limited to the cylindrical shape, and may be an elliptic tubular shape, a rectangular tubular shape, or the like.

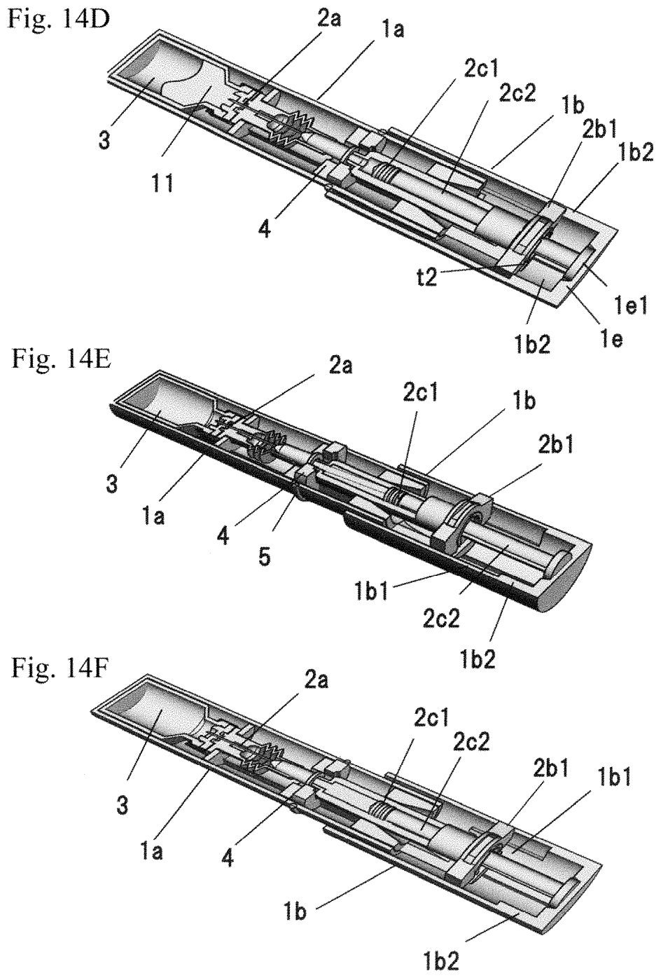

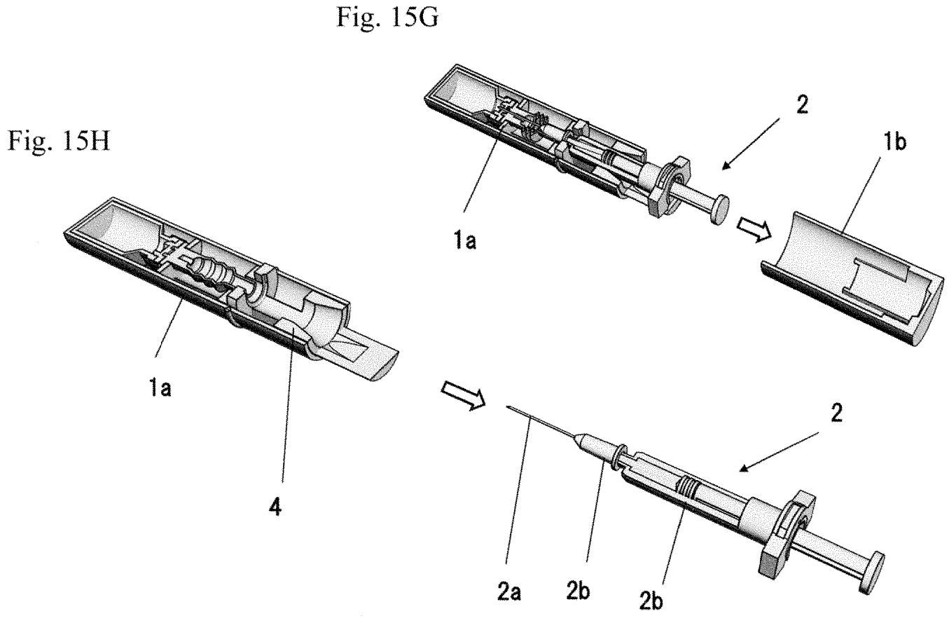

Next, the use of the two-component-mixing prefilled syringe kit according to the present invention is described in details. FIG. 13A to FIG. 14F are a perspective drawings showing an injection solution preparing process by the prefilled syringe kit of FIG. 1, and FIG. 15H is a perspective drawing showing take out of the syringe from the prefilled syringe kit of FIG. 1.

First, in the initial state (FIG. 13A), when the large-diameter cylindrical portion 1b of the accommodating case 1 is pushed into the small-diameter cylindrical portion 1a along the axial direction, the toroidal thin plate portion 1c is broken and the large-diameter cylindrical portion 1b moves along the axial direction so as to cover the small-diameter cylindrical portion 1a (FIG. 13B). At this time, the side surface t1 of the rib 1b1 of the large-diameter cylindrical portion 1b is in contact with the rear edge surface of the flange portion 2b1 of the syringe barrel 2b, so that the syringe barrel 2b also moves in the same direction. Then, the tip of the injection needle 2a penetrates through the needle cap 2d and the seal member 3a to reach the inside of the container 3.

At the same time, the holding member 4 that fixes and supports the syringe barrel 2b is pushed toward the container 3, and as described by FIGS. 11A-11C and FIGS. 12A, 12B, the pushing portion 4e of the holding member 4 in a state of sandwiching the flange portion 2d4 of the needle cap 2d is in contact with the edge surface of the container 3, and the tubular portion 2d5 of the front portion 2d3 of the needle cap 2d is pushed against the seal member 3a, and this state is maintained.

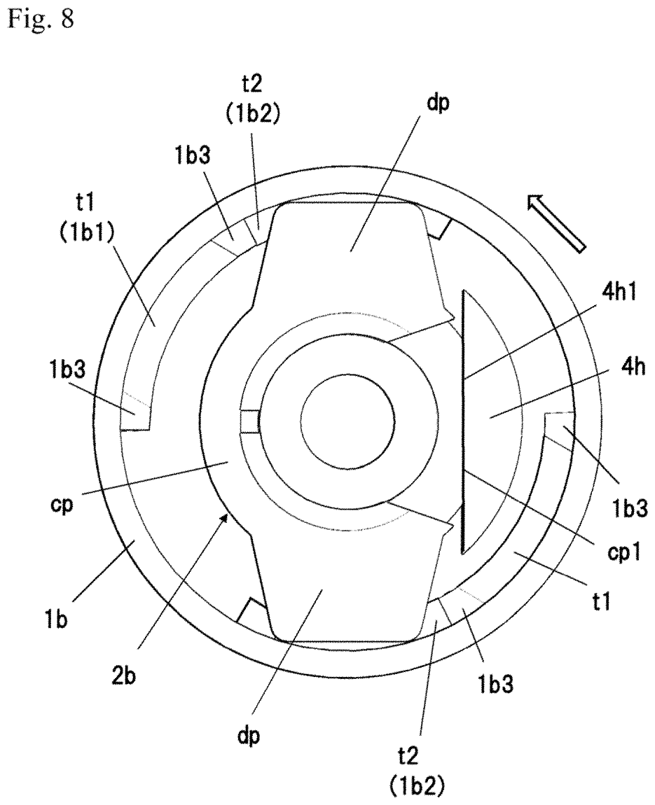

Next, the large-diameter cylindrical portion 1b is slightly pulled back in the axial direction with respect to the small-diameter cylindrical portion 1a, and then the large-diameter cylindrical portion 1b is rotated about 60 degrees to the right direction (in FIG. 8, to left rotation) around the axial center with respect to the syringe barrel 2b as shown in FIG. 8, and the two trapezoidal portions dp of the flange portion 2b1 are positioned so as to overlap the second rib 1b2 in the axial direction view (FIG. 13C). FIG. 8 is a section arrow drawing showing the position of the accommodating case 1 rotated with respect to the syringe barrel 2b. When the large-diameter cylindrical portion 1b is pushed in the axial direction against the small-diameter cylindrical portion 1a at the rotational position of FIG. 8, the movement is stopped at a position where the side surface t2 of the second rib 1b2 comes into contact with the trapezoidal portion dp of the flange portion 2b1 (FIG. 14D). At this time, the driving rod 2c2 that is in contact with the concave portion 1e1 of the wall 1e of the large-diameter cylindrical portion 1b is pushed, and the plunger 2c1 connected to the driving rod 2c2 is pushed into the syringe barrel 2b so that an air in the syringe barrel 2b is injected into the container 3, and the inside of the container 3 becomes in pressurized state.

Subsequently, when the large-diameter cylindrical portion 1b pushing the plunger 2c1 into the syringe barrel 2b is released to free, the pressurized air injected in the container 3 returns into the syringe barrel 2b together with the solution liquid 11 in the container 3, and the plunger 2c1 is pushed back. At the same time, the large-diameter cylindrical portion 1b is also pushed back by the driving rod 2c2 (FIG. 14E). Then, the solution liquid 11 that has flowed into the syringe barrel 2b is mixed with the powder drug 10 to dissolve it, and an injection solution is produced.

Finally, the large-diameter cylindrical portion 1b is slightly pushed against the small-diameter cylindrical portion 1a in the axial direction to move the plunger 2c1, and the bubbles remaining in the syringe barrel 2b are discharged from the tip of the injection needle 2a into the container 3, whereby the preparation of the injection solution is completed (FIG. 14F). After that, the large-diameter cylindrical portion 1b is moved to the rear side in the axial direction to be removed from the small-diameter cylindrical portion 1a (FIG. 15G), and the syringe 2 is pulled out in the axial direction to be taken out from the holding member 4 (FIG. 15H). At this time, since the pushing portion 4e of the holding member 4 pushes the flange portion 2d4 of the needle cap 2d against the edge surface of the container 3 (see FIGS. 12A, 12B), the needle cap 2d fixed to the outer circumference of the flange portion 2a1 of the injection needle 2a is peeled off and remains in the holding member 4 side, and the syringe 2 with the injection needle 2a exposed is taken out.

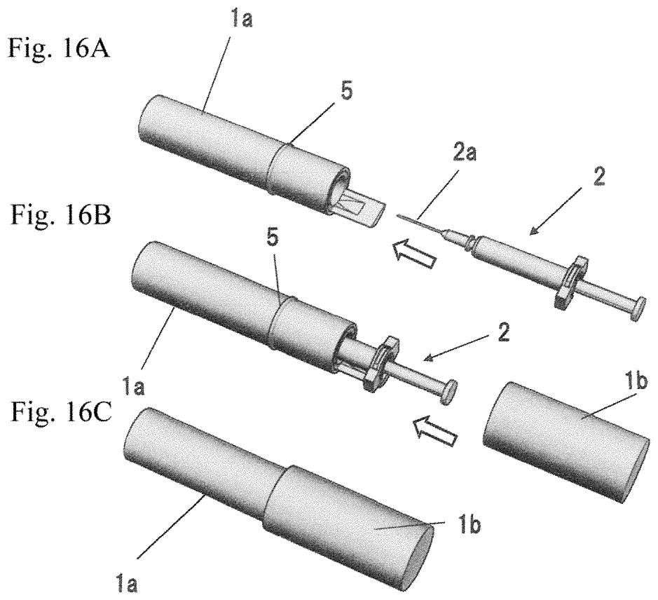

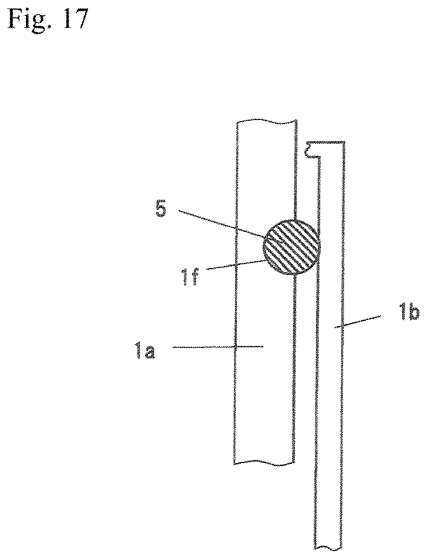

Next, the disposal of the used syringe is described. FIGS. 16A-16C are perspective drawings showing a disposal preparation process of a used syringe, and FIG. 17 is an enlarged schematic section of a part of the accommodating case 1 after completion of the disposal preparation process.

First, the used syringe 2 is inserted from the injection needle 2a side into the end opening of the small diameter-cylindrical portion 1a along the axial direction (FIG. 16A). Subsequently, the large-diameter cylindrical portion 1b is moved along the axial direction to a position where the large-diameter cylindrical portion 1b completely covers the rear side of the syringe 2 popping out from the end opening of the small-diameter cylindrical portion 1a (FIGS. 16B, 16C). At this time, as shown in FIG. 17, the large-diameter cylindrical portion 1b is moved to a position where the inner circumferential wall of the large-diameter cylindrical portion 1b is in contact with the O-ring 5 provided on the entire outer circumference of the small-diameter cylindrical portion 1a. As described in FIGS. 6A-6C, since the protrusion amount W1 of the O-ring 5 from the outer circumferential surface of the small-diameter cylindrical portion 1a is larger than the difference W2 of the positions in the radial direction between the outer circumferential surface of the small-diameter cylindrical portion 1a and the inner circumferential surface of the large-diameter cylindrical portion 1b, the O-ring 5 is crushed evenly over the entire circumference, and the inside of the accommodating case 1 is sealed from the outside by the O-ring 5. As a result, the injection solution remaining inside the syringe 2 does not leak outside.

Next, another embodiment of the two-component-mixing prefilled syringe kit according to the present invention is described. FIG. 18 is a partially enlarged section of the prefilled syringe kit according to the first another embodiment of the present invention.

In this another embodiment, a cylindrical rubber adapter 7 is attached to the surface of the seal member 3a, and an insertion portion 2d8 that can be fitted into the inner surface portion of the adapter 7 to be connected is provided at the front portion 2d7 of the needle cap 2d, and the difference is that the adapter 7 and the insertion portion 2d8 form a space sealed from the outside on the surface side of the seal member 3a. That is, the space-forming body 100 is constituted by the adapter 7 and the insertion portion 2d8 of the needle cap 2d.

The adapter 7 has a toroidal protrusion portion 7a protruding inward at the end on the needle cap 2d side, and has a disk-shaped flange portion 7b protruding outside at the end on the container 3 side and a cylindrical leg portion 7c inclining inside toward the surface of the seal member 3a. The insertion portion 2d8 of the needle cap 2d is formed in a tapered columnar shape whose outer diameter decreases toward the tip, and has a groove portion 2d9 having a diameter smaller than that of the protrusion porti0on 7a at the base side. As a result, the insertion portion 2d8 is pushed into close contact with the inner surface of the adapter 7, and the protrusion portion 7a of the adapter 7 is fitted into the groove portion 2d9 of the insertion portion 2d8 so that the insertion portion 2d8 is not taken off from the adapter 7.

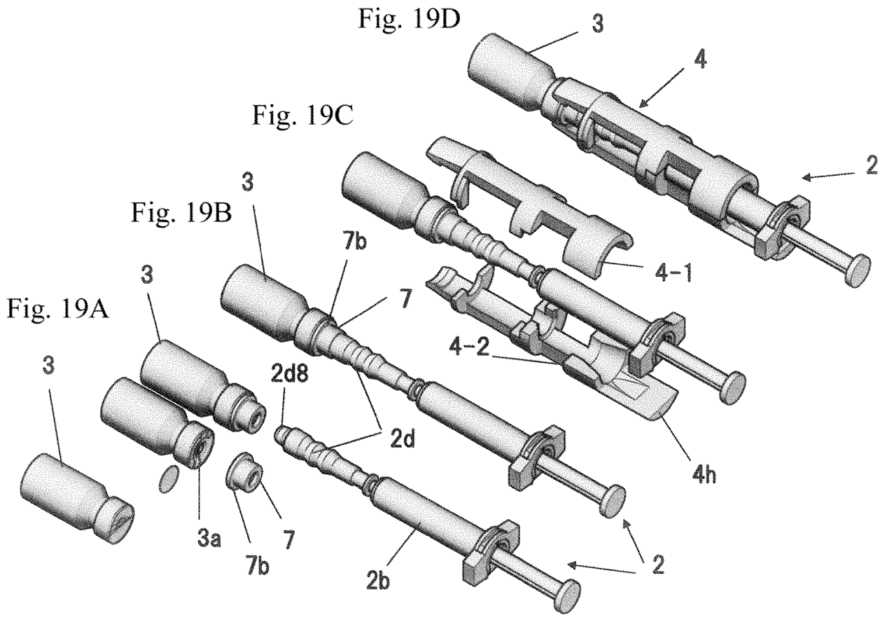

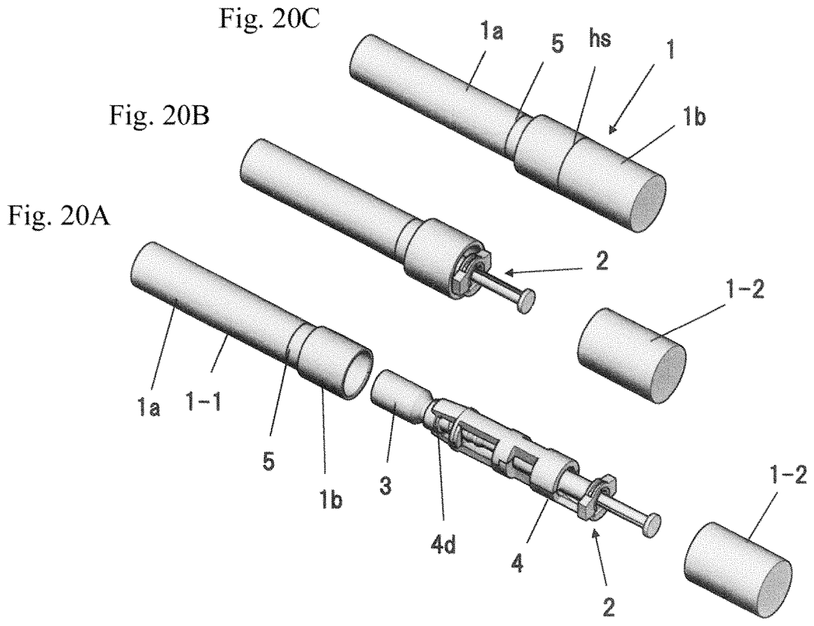

Next, the assembly of the two-component-mixing prefilled syringe kit of the first another embodiment is described. FIGS. 19A-19D are perspective drawings showing an assembling process of the syringe to the holding member, and FIGS. 20A-20C are perspective drawings showing an assembling process of the prefilled syringe kit shown in FIG. 18.

In assembling the syringe 2 to the holding member 4, first, the protective film of the seal member 3a of the container 3 is removed, and the adapter 7 is temporarily fixed to the surface of the seal member 3a with an adhesive (FIG. 19A). At this time, the outer side surface of the leg portion 7c of the adapter 7 closely faces the surface of the seal member 3a of the container 3. Next, the insertion portion 2d8 of the tip of the needle cap 2d of the syringe 2 in which the powder drug 10 is filled in the syringe barrel 2b in advance is inserted into the adapter 7 to integrally connect the container 3 and the syringe 2 (FIG. 19B). Next, the syringe 2 with the container 3 connected is set from above to the semi-cylindrical member 4-2 with the inside facing upward. At this time, the position is arranged so that the straight outer circumferential portion cp1 of the flange portion 2b1 comes into contact with the rotation stop portion 4h of the holding member 4 (see FIG. 7). Also, the semi-cylindrical member 4-1 with the inside facing downward is covered over the syringe 2 with the container 3 connected so as to be integrally connected with the semi-cylindrical member 4-2 (FIGS. 9C, 9D). As a result, the holding member 4 is formed by the semi-cylindrical members 4-1 and 4-2 that are integrally connected, and the syringe 2 connected with the container 3 is assembled to the holding member 4.

In assembling the prefilled syringe kit, the syringe 2 assembled to the holding member 4 is inserted into the end opening of the case component 1-1 at the large-diameter cylindrical portion 1b side with the container 3 side front (FIG. 20A). Next, the case component 1-2 is covered so as to cover the rear side of the syringe 2 protruding from the opening of the case component 1-1, and the opening ends of both the case components 1-1, 1-2 are connected so that both step portions 1b4, 1b5 are fitted to each other (FIGS. 20B, 20C). Finally, the step portions 1b4, 1b5 of the both case components 1-1, 1-2 connected are joined by heat to form a joint hs, and the inside of the case 1 is sealed.

Next, the injection solution preparing operation by the two-component-mixing prefilled syringe kit of the first another embodiment is described. FIGS. 21A and 21B are sections for explaining an injection solution preparing operation by the prefilled syringe kit of FIG. 18.

When the syringe barrel 2b is pushed to the container 3 side, the bellows portion 2d2 of the needle cap 2d contracts, and the injection needle 2a pierced up to the midpoint of the insertion portion 2d8 of the needle cap 2d penetrates the remaining portion of the insertion portion 2d8 and the seal member 3a and projects into the inside of the container 3 (FIG. 18 and FIG. 21A). At this time, the holding member 4 holding the syringe barrel 2b is also pushed toward the container 3 side, and the hook portion 4d1 of the movable piece 4d that is in contact with the circumferential edge corner portion of the stopper 3b of the container 3 at the inclined surface t3 slides and moves outside, as it moves, the support portion 4d2 bends outward, and the hook portion 4d1 moving outside passes by sliding on the side surface of the stopper 3b. At the same time, the pushing portion 4e of the holding member 4 pushes the flange portion 7b of the adapter 7 against the front surface of the stopper 3b of the container 3. Then, the hook portion 4d1 pushed out to the outside returns to the inside and is hanged by the step portion 3b1 on the base side of the stopper 3b, and the holding member 4 is mechanically connected to the container 3 at this position. As a result, the state in which the injection needle 2a projects into the container 3 and the state in which the flange portion 7b of the adapter 7 is pushed against the front surface of the stopper 3b of the container 3 are maintained.

Next, the plunger 2c1 is pushed with a finger to send the air in the syringe barrel 2b into the container 3 to increase the pressure in the container 3, and then the finger is released from the plunger 2c1 to be free, the liquid 11 in the container 3 is suctioned to the inside of the syringe barrel 2b through the injection needle 2a by internal pressure, mixed with the powder drug 10 inside the syringe barrel 2b to produce an injection solution, and the plunger 2c1 is returned to its original position. Finally, the syringe barrel 2b is grasped with fingers, the injection needle 2a is pulled out from the container 3, and is pulled back to the inside of the needle cap 2d (FIG. 21B). At this time, a part of the liquid 11 remaining in the container 3 may overflow onto the surface of the seal member 3a through the needle piercing place st of the seal member 3a (see FIGS. 12A, 12B). But, since the space surrounded by the seal member 3a and the adapter 7 and the insertion portion 2d8 of the needle cap 2d is sealed from the outside, the liquid 11a overflowing from the container 3 does not leak to the outside.

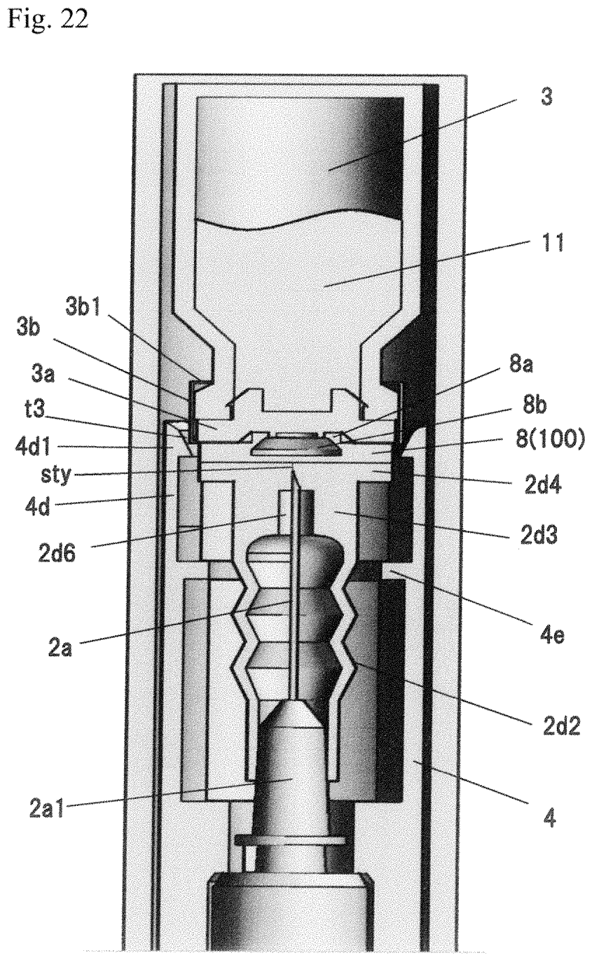

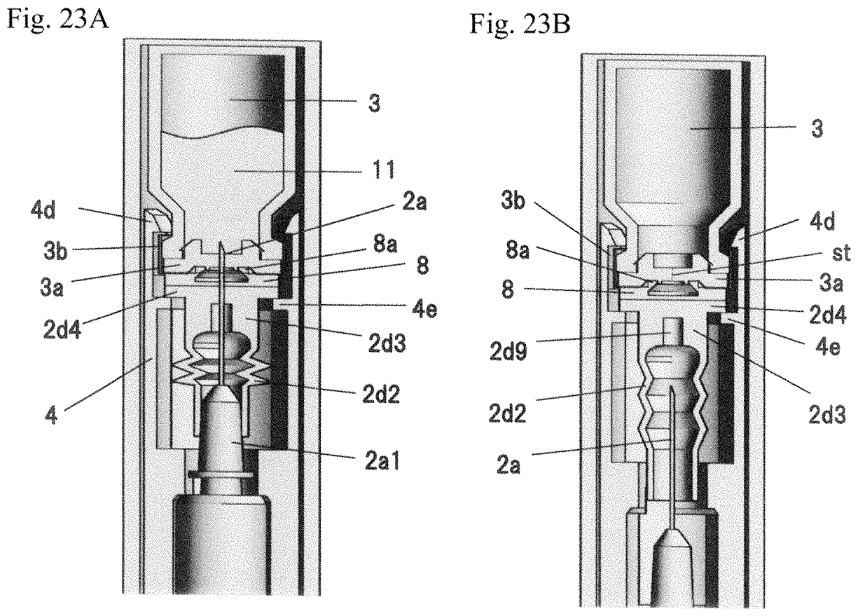

Next, a second another embodiment of the two-component-mixing prefilled syringe kit of the present invention is described. FIG. 22 is a partially enlarged section of the prefilled syringe kit according to the second another embodiment of the present invention, and FIGS. 23A, 23B are sections for explaining an injection solution preparing operation by the prefilled syringe kit of FIG. 22.

In this second another embodiment, the difference is that a disk-shaped rubber adapter 8 attached to the surface of the seal member 3a has a cavity 8b opened only on the seal member 3a side, and the adapter 8 is attached to the surface of the seal member 3a, whereby a space sealed from the outside is formed on the surface side of the seal member 3a. That is, the space-forming body 100 is constituted by the adapter 8.

The adapter 8 is formed flat plate-like at the edge portion on the needle cap 2d side, and has a cylindrical leg portion 8a inclined inside toward the surface of the seal member 3a at the edge portion on the seal member 3a side. The needle cap 2d has a front portion 2d3 whose side facing the adapter 8 is formed a flat surface, and the front portion 2d3 is provided with a toroidal plate-shaped flange portion 2d4.

Next, the injection solution preparing operation by the two-component-mixing prefilled syringe kit of another embodiment is described. FIGS. 23A and 23B are sections for explaining an injection solution preparing operation by the prefilled syringe kit of FIG. 22.

When the syringe barrel 2b is pushed to the container 3 side, the bellows portion 2d2 of the needle cap 2d contracts, and the injection needle 2a pierced up to the middle of the front portion 2d3 of the needle cap 2d penetrates the remaining part of the front portion 2d3, the adapter 8 and the seal member 3a to project to the inside of the container 3 (FIG. 22 and FIG. 23A). At this time, the holding member 4 holding the syringe barrel 2b is also pushed to the container 3 side, and the hook portion 4d1 of the movable piece 4d that is in contact with the circumferential edge corner of the stopper 3b of the container 3 at the inclined surface t3 slides and moves outside, as it moves, the support portion 4d2 bends outward, and the hook portion 4d1 moving outside passes by sliding on the side surface of the stopper 3b. At this time, the pushing portion 4e of the holding member 4 pushes the flange portion 2d4 of the needle cap 2d and the circumferential edge portion of the disk-shaped adapter 8 with overlapped, against the front surface of the stopper 3b of the container 3. Then, the hook portion 4d1 pushed out to the outside returns to the inside and is hanged by the step portion 3b1 on the base side of the stopper 3b, and the holding member 4 is mechanically connected to the container 3 at this position. As a result, the state in which the injection needle 2a projects into the container 3 and the state in which the pushing portion 4e of the holding member 4 pushes the flange portion 2d4 of the needle cap 2d and the circumferential edge portion of the disk-shaped adapter 8 with overlapped against the front surface of the stopper 3b of the container 3 are maintained.

Next, the plunger 2c1 is pushed with a finger to send the air in the syringe barrel 2b to the container 3 to increase the pressure in the container 3, and then the finger is released from the plunger 2c1 to be free, the liquid 11 in the container 3 is suctioned to the inside of the syringe barrel 2b through the injection needle 2a by internal pressure, and is mixed with the powder drug 10 inside the syringe barrel 2b to produce an injection solution, and the plunger 2c1 is returned to its original position. Finally, the syringe barrel 2b is grasped with fingers so that the injection needle 2a is pulled out from the container 3, and is pulled back to the inside of the needle cap 2d (FIG. 23B). At this time, a liquid 11a being a part of the liquid 11 remaining in the container 3 may overflow to the surface of the seal member 3a through the needle piercing place st of the seal member 3a (see FIGS. 12A, 12B). But, since the cavity 8b of the adapter 8 communicating with the surface of the seal member 3a is sealed from the outside, the liquid 11a overflowing from the container 3 does not leak to the outside.

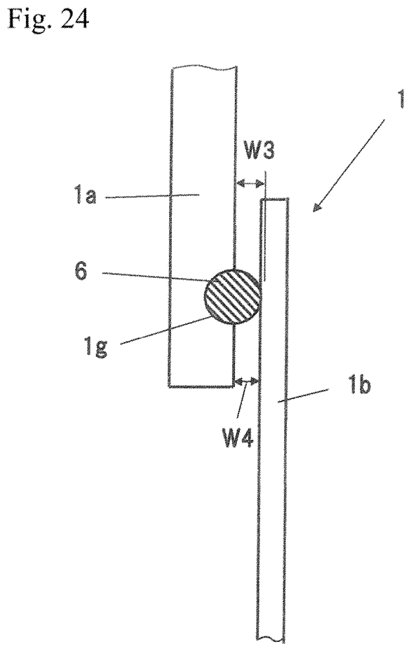

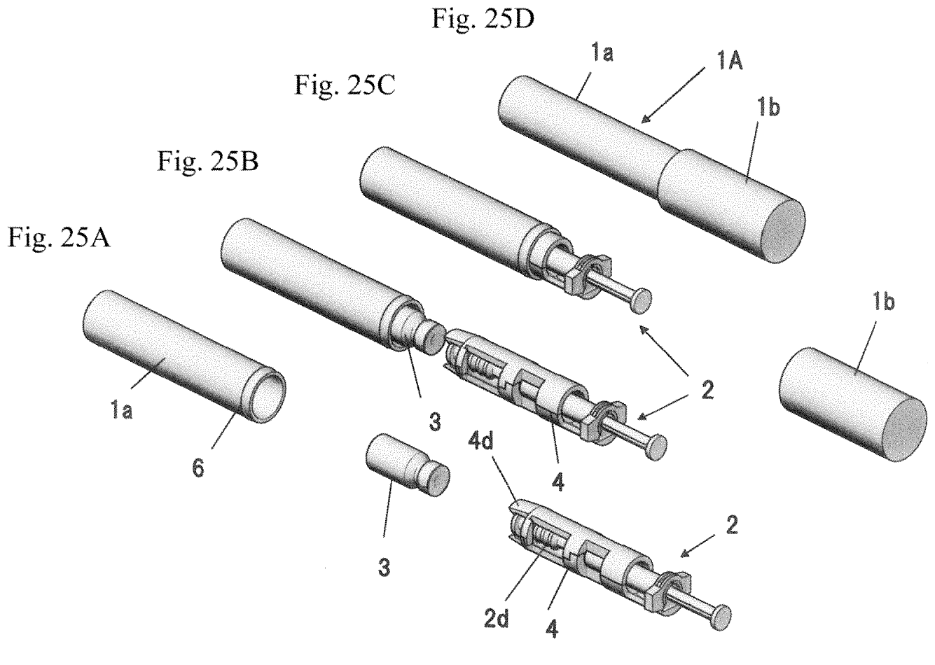

Next, a third another embodiment of the two-component-mixing prefilled syringe kit according to the present invention is described. FIG. 24 is a partially enlarged section showing the structure of a prefilled syringe kit according to a third another embodiment of the present invention, and FIGS. 25A, 25B are a perspective drawings showing an assembly process of the prefilled syringe kit shown in FIG. 24.

In the third another embodiment, the structure of the accommodating case 1A is different from that of the above-described prefilled syringe kit, and the other structures are the same.

The accommodating case 1A does not have the structure in which the small-diameter cylindrical portion 1a and the large-diameter cylindrical portion 1b are connected by the thin plate portion 1c as in the above-described embodiment, but has a structure in which in a state where the small-diameter cylindrical portion 1a and the large-diameter cylindrical portion 1b are mutually axially aligned, the O-ring 6 is arranged between the outer circumferential portion of one end of the small-diameter cylindrical portion 1a and the inner circumferential portion of one end of the large-diameter cylindrical portion 1b.

A concave groove 1g having a semicircular section is formed over the entire circumference on the outer circumference of the small-diameter cylindrical portion 1a, and an O-ring 6 is fitted in the concave groove 1g. The protrusion amount W3 of the O-ring 6 from the outer circumferential surface of the small-diameter cylindrical portion 1a is set to a value larger than half W4 of the difference between the inner diameter of the large-diameter cylindrical portion 1b and the outer diameter of the small-diameter cylindrical portion 1a (W3>W4). Therefore, the O-ring 6 is crushed evenly over the entire circumference, and the inside of the accommodating case 1A is sealed from the outside. Also, the concave groove 1g is provided closer to one end of the small-diameter cylindrical portion 1a, comparing with the concave groove 1f of the above embodiment.

In the assembling process of the prefilled syringe of the third another embodiment, first, the container 3 is inserted into the end opening of the small-diameter cylindrical portion 1a from the bottom side (FIG. 25A). Subsequently, the holding member 4 set with the syringe 2 is inserted into the small-diameter cylindrical portion 1a with the movable piece 4d facing forward (FIG. 25B). Next, the large-diameter cylindrical portion 1b is covered so as to cover the rear side of the syringe 2 protruding from the end opening of the small-diameter cylindrical portion 1a (FIG. 18C), and the large-diameter cylindrical portion 1b is moved in the axial direction to a position where the inner circumferential surface of the large-diameter cylindrical portion 1b is in contact with the O-ringed 6, so that a sealed accommodating case 1A is constituted (FIG. 18D).

In the use of the prefilled syringe of the third another embodiment, since the small-diameter cylindrical portion 1a and the large-diameter cylindrical portion 1b are not directly connected, except the first step of breaking and separating the small-diameter cylindrical portion 1a and the large-diameter cylindrical portion 1b is unnecessary, it is the same as the above-described embodiment.

The disposal of the used syringe by the prefilled syringe of the third another embodiment is the same as in the above embodiment (see FIGS. 14D, 14F). When the used syringe 2 is stored in the accommodating case 1A in another embodiment, the inner circumferential wall of the large-diameter cylindrical portion 1b is in contact with the O-ringed 6 provided on the entire of outer circumference of the small-diameter cylindrical portion 1a, whereby the inside of the accommodating case 1A is sealed from the outside by the O-ringed 6.

Next, another embodiment according to the present invention is described. First, in the above-described embodiment, the powder drug 10 is filled in the inside of the syringe barrel 2b as one component constituting the injection solution, and the liquid 11 (solution or dispersion liquid) is filled in the inside of the container 3 as the other component constituting the injection solution. However, the syringe barrel 2b may be filled with a solid drug such as granules instead of the powder drug.

Also, both the syringe barrel 2b and the container 3 may be filled with the liquid component. Specifically, the container 3 is filled with a liquid drug such as a vaccine, which has poor stability when premixed, and the syringe barrel 2b is filled with the solution. Alternatively, the syringe barrel 2b is filled with the liquid drug and the container 3 is filled with the solution. As a result, it is effective when it is necessary to fill a liquid drug having poor stability separately with a solution and to mix them when preparing an injection solution.

In the above-described embodiment, the tip of the injection needle 2a is held in a state of being pierced by the needle cap 2d (however, in a non-penetrating state), but it is also good that the tip of the injection needle 2a is held in a state of being separated from the needle cap 2d.

In the above-described embodiment, the holding member 4 holds the syringe barrel 2b so as to be movable along the barrel axis direction of the syringe barrel 2b, but it may be constituted that the holding member holds the container 3 so as to be movable along the barrel axis direction of the syringe barrel 2b.
In the above-described embodiment, the container 3 is constituted by a vial, but may be constituted by a container other vial.

The embodiments disclosed this time are to be considered as illustrative in all points and not restrictive. The scope of the present invention is shown not by the above description but by the scope of the claims, and is intended to include meanings equivalent to the scope of the claims and all modifications within the scope.

### EXPLANATION OF SYMBOLS

1 accommodating case, 1A accommodating case, 1a small-diameter cylindrical portion, 1b large-diameter cylindrical portion, 1b1 rib, 1b2 second rib, 1b3 third rib, 1b4 step portion, 1b5 step portion, 1c thin plate portion, 1d wall, 1e wall, 1e1 concave portion, 1f concave groove, 1g concave groove, 1-1 case component, 1-2 case component, 2 syringe, 2a injection needle, 2a1 flange portion, 2b syringe barrel, 2b1 flange portion, 2c1 plunger, 2c2 driving rod, 2d needle cap, 2d1 cylindrical portion, 2d2 bellows portion, 2d3 front portion, 2d4 flange portion, 2d5 tubular portion, 2d6 cavity, 2d7 front portion, 2d8 insertion portion, 2d9 groove portion, D height, H inner diameter, V space (sealed space), 3 container, 3a seal member, 3b stopper, 3b1 step portion, 3c bottom portion, 3d flange portion, 4 holding member, 4a toroidal rib, 4b toroidal step portion, 4c cylindrical guide, 4d movable piece, 4d1 hook portion (lock part, claw body), 4d2 support portion, 4e pushing portion, 4a1 step portion, 4a2 step portion, 4e1 convex portion, 4e2 step portion, 4f cylindrical guide, 4-1 Semi-cylindrical member, 4-2 Semi-cylindrical member, 5 O-ring, 6 O-ring, 7 adapter, 7a protrusion portion, 7b flange portion, 7c leg portion, 8 adapter, 8a leg portion, 8b cavity, 10 powder drug, 11 liquid (solution or dispersion liquid) , 50 mount portion, 100 space-forming body, hs joint, st needle piercing place, sty expected piercing place, t1 side surface, t2 side surface, t3 inclined surface

1. A two-component-mixing prefilled syringe kit, comprising;
a syringe barrel in which an injection needle is attached to one end, a plunger is slidably inserted from an opening of the other end, and a solid component or a liquid component constituting an injection solution is filled inside;
a container in which an outlet is sealed with a seal member that can be pierced by the injection needle, and a liquid component constituting the injection solution is filled inside;
a holding member for holding at least one of the syringe barrel or the container so as to be movable along the barrel axis direction of the syringe barrel with the tip of the injection needle facing the outlet of the container;
wherein, it is characterized that a space-forming body which forms a space facing the surface of the seal member with a space sealed from the outside is arranged so as to be in close contact with the surface of the seal member, and the space-forming body is formed of a material which can be pierced by the injection needle at a portion facing the tip of the injection needle along the barrel axis direction of the syringe barrel.

2. The two-component-mixing prefilled syringe kit as defined in claim 1, wherein;
a volume of a space surrounded by the surface of the seal member and the space-forming body is in the range of 0.001 ml to 0.03 ml.

3. The two-component-mixing prefilled syringe kit as defined in claim 1, further comprising:
a needle cap which is formed of a material that can be pierced by the injection needle to cover the injection needle from the tip side, and fixed at base end side to a base portion of the injection needle or an outer surface of the syringe barrel, wherein
the needle cap has a tubular portion in which a tubular axis direction is along to the direction toward the seal member and an expected piercing place by the injection needle is located at the inner side, at a front portion facing the seal member,
the space-forming body is constituted by the front portion of the needle cap and the tubular portion in close contact with the surface of the seal member.

4. The two-component-mixing prefilled syringe kit as defined in claim 3, wherein;
the needle cap has a flange portion in a direction intersecting with the barrel axis direction of the syringe barrel, on the outer side surface of the front portion;
the holding member comprises a mount portion for mounting the syringe barrel, a pushing portion for pushing the flange portion against the container when moves to the container side along the barrel axis direction of the syringe barrel, and a lock part for holding a state where the pushing portion pushes the flange portion against the container.

5. The two-component-mixing prefilled syringe kit as defined in claim 3, wherein;
the length of the tubular portion along the tubular axis direction is less than 4 mm.

6. The two-component-mixing prefilled syringe kit as defined in claims 1, further comprising:
a needle cap which is formed of a material that can be pierced by the injection needle to cover the injection needle from the tip side, and fixed at the base end side to a base portion of the injection needle or the outer surface of the syringe barrel;
a tubular container adapter which has a tube axis coinciding with the barrel axis direction of the syringe barrel, and fixed in close contact with the surface of the seal member at an one end in the tube axis direction, wherein
the needle cap has an insertion portion inserted along the tube axis direction of the container adapter from the other end side in the tube axis direction so as to be in close contact with the inner surface of the container adapter, at a front portion facing the seal member;
the space-forming body is constituted by the container adapter and the insertion portion of the needle cap.

7. The two-component-mixing prefilled syringe kit as defined in claim 6, wherein;
the container adapter has a flange portion in a direction intersecting with the barrel axis direction of the syringe barrel, on the outer side surface of the seal member side,
the holding member has a mount portion for mounting the syringe barrel, and a pushing portion for pushing the flange portion against the container when the syringe barrel mounted on the mount portion is moved along a barrel axis direction to the container side, and a lock part for holding the state where the pushing portion pushes the flange portion against the container.

8. The two-component-mixing prefilled syringe kit as defined in claims 1, further comprising;
a needle cap which is formed of a material that can be pierced by the injection needle to cover the injection needle from the tip side, and fixed at a base end side to a base portion of the injection needle or the outer surface of the syringe barrel;
a container adapter having a tubular portion which is fixed in close contact with the surface of the seal member at one end in the tube axial direction, and a flat plate-shaped main body portion which covers the opening on the other end side of the tubular portion, wherein
the main body portion of the container adapter is formed of a material pierceable by the injection needle at a portion facing the seal member,
the space-forming body is constituted by the container adapter.

9. The two-component-mixing prefilled syringe kit as defined in claim 8, wherein;
the container adapter has a flange portion in a direction intersecting with the barrel axis direction of the syringe barrel on the outer surface of the seal member side,
the needle cap has a flange portion in a direction intersecting with the barrel axis direction of the syringe barrel on the outer side surface of the front side,
the holding member has a mount portion for mounting the syringe barrel, and a pushing portion that pushes the two flange portions of the needle cap and the container adapter in overlapped state against the container when moving to the container side along the barrel axis direction of the syringe barrel and the lock part that holds the state where the pushing portion pushes the two flange portions against the container.

10. The two-component-mixing prefilled syringe kit as defined in claim 8, wherein;
the length of the tubular portion along the tube axis direction is less than 4 mm.

11. The two-component-mixing prefilled syringe kit as defined in claim 4, wherein;
it is characterized that the lock part is a claw body which can be fitted to a concave portion or a step portion on the outer surface of the container.

12. The two-component-mixing prefilled syringe kit as defined in claim 3, further comprising;
an accommodating case which accommodates the whole of the syringe barrel, the needle cap, the container and the holding member in a sealed state to the outside.

13. The two-component-mixing prefilled syringe kit as defined in claim 6, further comprising;
an accommodating case which accommodates the whole of the syringe barrel, the needle cap, the container adapter, the container and the holding member in a sealed state to the outside.

14. The two-component-mixing prefilled syringe kit as defined in claim 3, wherein;
the tip of the injection needle is held by the needle cap in a non-penetrated piecing state.
